(19) United States
(12) Patent Application Publication
(10) Pub. No.: US 2014/0100217 A1
(43) Pub. Date: Apr. 10, 2014

(54) STRUCTURAL MIMETICS OF PROLINE-RICH PEPTIDES AND THEIR USE

(71) Applicants: FORSCHUNGSVERBUND BERLIN E.V., BERLIN (DE); UNIVERSITAET ZU KOELN, KOELN (DE)

(72) Inventors: HANS-GUENTHER SCHMALZ, BRUEHL (DE); CÉDRIC MICHAEL REUTER, KOELN (DE); RONALD KUEHNE, BERLIN (DE); HARTMUT OSCHKINAT, BERLIN (DE); MATTHIAS MUELLER, BERLIN (DE); ROBERT OPITZ, BERLIN (DE)

(73) Assignees: FORSCHUNGSVERBUND BERLIN E.V., BERLIN (DE); UNIVERSITAET ZU KOELN, KOELN (DE)

(21) Appl. No.: 14/049,233

(22) Filed: Oct. 9, 2013

Related U.S. Application Data
(60) Provisional application No. 61/711,756, filed on Oct. 10, 2012.

(51) Int. Cl. C07D 487/04

(52) U.S. Cl. CPC C07D 487/04
USPC 514/212.07; 540/523

(57) ABSTRACT

The present invention provides a compound comprising a general formula 1:

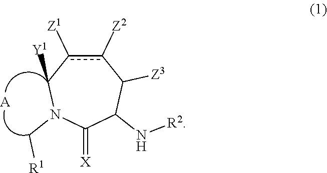

In general formula 1, X is at least one of O and S. A is a ring bridge. Y¹ is at least one of H, alkyl, fluoroalkyl, aryl and heteroaryl. Z¹, Z², Z³ are, individually or alternatively, at least one of H, carbonyl, OH, O-alkyl, O-acyl, N—R¹R² (where R¹ or R² are, individually or alternatively, at least one of H, alkyl, acyl, and sulfonyl), alkyl, acyl, fluoroalkyl, aryl, and heteroaryl. R¹ is at least one of alkyl, acyl, alkoxycarbonyl, aryloxycarbonyl, and aminocarbonyl. R² is at least one of H, alkyl, aryl, alkylcarbonyl, arylcarbonyl, alkoxycarbonyl, aminocarbonyl, aryloxycarbonyl, alkylsulfonyl, arylsulfonyl, aminoacyl and peptidyl. The present invention furthermore relates to the use of the compound as a pharmaceutical active compound, and to the use of the pharmaceutical active compound to treat bacterial diseases, neurodegenerative diseases and tumors.

### CROSS REFERENCE TO PRIOR APPLICATIONS

Priority is claimed to U.S. Provisional Patent Application No. 61/711,756, filed Oct. 10, 2012. The entire disclosure of said application is incorporated by reference herein.

### FIELD

The present invention relates to compounds that can, for example, be employed as structural mimetics of proline-rich peptides and accordingly are able to bind PRM binding domains (proline-rich motif binding domains) of proteins. The present invention furthermore relates to the use of these compounds as pharmaceutical active compounds and to the use of the pharmaceutical active compounds for the treatment of bacterial diseases, neurodegenerative diseases and tumors.

### BACKGROUND

Peptides and proteins are essential constituents of organisms with a multiplicity of different functions. While the proteins have biocatalytic functions (enzymes) and as such are important tissue constituents, the peptides fulfill important functions in the body, especially as hormones, neurotransmitters and neuromodulators. As a result of binding to membrane-bound receptors and cytophysiological secondary reactions mediated thereby, peptides influence cell-cell communication and control a multiplicity of vital processes such as metabolism, immune defense, digestion, respiration, sensation of pain, reproduction, behavior, electrolyte balance and more.

There is therefore the need in the state of the art to elucidate the exact relationships in the body and at the same time to provide a necessary basis for the treatability of pathogenic conditions. With an increasing understanding of biological processes at the molecular level, the interconnection of biology and chemistry has also increased, assisted by great advances in analytical processes and computer-aided theoretical methods. All these are important prerequisites for the successful identification of lead structures in active compound development. We are, however, still far removed from the actual aim, the simple and efficient de novo design of active compounds. A great amount of empirical research effort is much rather generally required to synthesize libraries of possible target substances starting from natural structures and to optimize these to a specific action. In addition to the high amount of time and enormous costs, it moreover often turns out that active compounds developed with the aid of computers only inadequately achieve the desired effect in real, very complex biological systems (e.g., man) or have intolerable side effects.

Against this background, the development, especially also of peptide or peptide-mimetic active compounds, remains a great challenge, even in synthetic terms, since in the diverse interdisciplinary teamwork (last but not least organic chemistry) with its possibilities and restrictions determines access to desired target molecules. As these must be synthesized in as few steps as possible and generally stereoselectively, newer and better synthesis methods are constantly needed to achieve this aim not only in the laboratory, but subsequently also with a view to industrial application.

### SUMMARY

An aspect of the present invention is to make compounds available that can be employed as mimetics for proline-rich peptides, in particular, for those favoring a PPII helix conformation. The proline-proline dipeptide units, in particular, with a PPII helix conformation, can, for example, function as ligands for “PRM binding domains” (PRM=proline-rich motifs).

In an embodiment, the present invention provides a compound of the general formula (1):

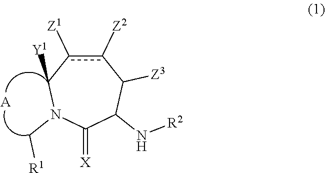

having a saturated or unsaturated central seven-membered ring, where,

X=O and/or S;

A=a ring bridge;

Y¹=H, alkyl, fluoroalkyl, aryl and/or heteroaryl;

Z¹, Z², Z³=H; carbonyl; OH; O-alkyl; O-acyl; N—R¹R² (where R¹ or R²=H, alkyl, acyl, sulfonyl); alkyl; acyl; fluoroalkyl; aryl and/or heteroaryl;

R¹=alkyl, acyl, alkoxycarbonyl, aryloxycarbonyl and/or aminocarbonyl (CONH₂, CONHR, CONH-peptidyl, (with R)); and

R²=H, alkyl, aryl, alkylcarbonyl, arylcarbonyl, alkoxycarbonyl, aminocarbonyl, aryloxycarbonyl, alkylsulfonyl, arylsulfonyl, aminoacyl and/or peptidyl.

### BRIEF DESCRIPTION OF THE FIGURES

The present invention is described in greater detail below on the basis of embodiments and of the Figures in which:

FIG. 1 shows the crystal structure of the bicycle 117;

FIG. 2 shows the ¹H-NMR spectrum of the diproline component 85 (500 MHz, CDCl, room temperature); and

FIG. 3 shows the VASP-EVH1 domain with ₃₃₂SFEFPₚₚPTEDEL₃₄₄ peptide ligand (molecular modeling depiction).

### DETAILED DESCRIPTION

The compounds according to the present invention surprisingly do not have the disadvantages of the prior art. In addition to the abovementioned disadvantages of the prior art, the use of peptide active compounds as medicaments was thus far restricted by a number of further factors including:
1) Low metabolic stability due to proteolysis in the gastrointestinal tract and in the serum;
2) Poor absorption after oral ingestion, especially on account of the high molecular mass;
3) Rapid excretion by the liver and kidneys; and
4) Lack of selectivity due to interaction with different receptors.

The compounds according to the present invention can surprisingly be employed as mimetics that are in particular able, as artificially produced substances, to exert the function of a receptor ligand and to imitate (agonist) or to block (antagonist) the biological effect of a peptide. Although the principle of the provision of peptide mimetics is known, only very few examples of PPII structural mimetics have been disclosed, which, however, have numerous disadvantages. Even though there were different approaches to replace the structural motif of the PPII helix completely or partially by synthetic analogs, with the known structures it was not possible or only possible to a very limited extent to investigate their interaction with different protein domains. The prior art also teaches a person skilled in the art that the interaction of proline-rich helixes with different protein domains has no particular biological significance. The present invention shows that this interaction or its modification accompanies numerous diseases such as bacterial or viral infections, neurodegenerative diseases or the formation of tumors. Numerous mimetics known in the prior art can only be made available with the aid of catalysts which, however, can often only be removed from the system with difficulty or not at all. The known products also do not have high storage stability and are often contaminated with products that are necessary for synthesis, which in particular would make impossible or difficult their use in medicine. The binding affinity of the mimetics known is also inadequate.

The interaction of peptide ligands with protein receptors plays an important role in the regulation of biological processes and depends crucially on the peptide geometry. Under physiological conditions, the conformation of a linear peptide due to rotation around individual bonds is in a dynamic equilibrium, which depends on the pH and on the temperature. This leads to the biological reactive conformation only being present to a low percentage.

The conformation of the peptide backbone is customarily described by the three angles φ (phi), ψ (psi) and ω (omega). On account of the partial double bond character, the peptide bond is hindered in its rotation and has a planar geometry, which leads to two primary conformations: the trans- and the cis-peptide bond with ω=180° or ω=0°, the trans-conformation being energetically more favorable and therefore predominating.

Therefore, to a first approximation, the torsion angles φ and ψ of the amino acid residues suffice for the conformational description of the peptide backbone. The angle φ, which describes the rotation along the N—C_{α} bond, is defined by the four atoms C(═O)—N—C_{α}—C(═O). In the same manner, N—C_{α}—C(═O)—N defines the angle ψ, which describes the rotation around the C_{α}—C(═O) bond. Although a large number of different combinations of φ and ψ is theoretically possible, certain conformations generally exist in peptides depending on size, polarity and charge of the side chains, which leads to the formation of the known secondary structures such as α-helix, β-pleated sheet, β-turn etc.

### The Amino Acid Proline as a Constituent of Peptides

Among the 20 naturally occurring amino acids, proline assumes a special position as the only secondary amino acid. Owing to the cyclization of the α-side chain to the amide nitrogen, as part of the five-membered ring, the torsion angle φ=(−65±15°) is relatively restricted. The peptide consequently has fewer degrees of rotational freedom. The double alkylation of the nitrogen on the one hand has the result that the otherwise customary amide proton (in the peptide backbone) is missing and proline is therefore eliminated as a hydrogen bridge donor, on the other hand, the carbonyl group is particularly electron-rich and therefore a better hydrogen bridge acceptor than in other amino acids. Owing to these geometric and electronic properties, proline cannot stabilize an α-helix (“α-helix breaker”) and also forms no β-pleated sheet structure (“β-pleated sheet breaker”) but is, for example, to be encountered in other typical secondary structures: the “β-turns” and the polyproline helix (PPII helix).

### Proline-Rich Motifs and the PPII Helix as a Secondary Structure

Proline-rich amino acid sequences are often found in peptides that are involved in intracellular signal transduction processes. These are sequences in which proline occurs exclusively or predominantly (often four and more proline units in a row).

The characteristic secondary structure, the polyproline helix or short PPII helix is thereby induced, under which is understood an extended left-handed helix with the torsion angles φ=−78° and ψ=+146° of the peptide backbone. As a consequence, a pseudo-C₃ rotational symmetry around the helix axis with exactly three proline radicals per rotation in the cross section results, which is why in proline-rich sequences the proline radicals repeat, for example, at least with a periodicity of three (e.g., PxxPxxP or PPxPPxPPx).

The proline side chains and the carbonyl groups of the peptide backbone are in this way exposed to the solvent at regular intervals. Due to the absence of intermolecular hydrogen bridges, the carbonyl groups are suitable for entering into intermolecular hydrogen bridge bonds to receptor proteins.

The structural motif of the PPII helix can, however, also be induced if proline is not exclusively present. The amino acid Glu occurs frequently in PPII helices and in their vicinity, but Gln, Arg, Ala, Leu, Ser, Asp and His are also found. The binding mode between domain and ligand determines which proline positions should be strictly conserved, and which can optionally be replaced by other amino acids.

In an embodiment of the present invention, this is selected such that A has 5 or 6 ring atoms, the ring members represented by A being selected from the group comprising C, O, S and/or N atoms. Of course, the ring bridge A can also be chosen such that a 4-, 5- or 6-membered ring results, the ring members consisting of —CH₂—, —O—, —S— and —N—R where R=H, alkyl, or acyl.

In an embodiment of the present invention, the compound has the general formula (2):

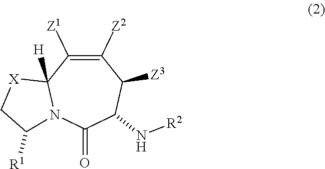

with Z¹, Z², Z³ being indicated for general formula (1) with the configuration shown in general formula (2);

with R¹, R²=alkyl, acyl, hetaryl and/or sulfonyl; and

with X=—CH₂—, —O—, —S— and/or —NH—R.

In an embodiment of the present invention, the compound has the general formula (3):

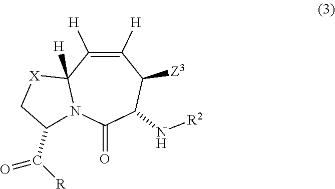

with Z³=H; carbonyl; OH; O-alkyl; O-acyl; N—R¹R² (where R¹ or R²=H, alkyl, acyl, sulfonyl); alkyl; acyl; fluoroalkyl; aryl and/or heteroaryl;

X=—CH₂—, —O— and/or —S—;

with R=NH—R″, —O—R″, where R″=peptidyl, substituted alkyls and/or hetaryl; and

with R²=acyl, peptidyl and/or sulfonyl.

In the formula 3, R² can, for example, be a peptidyl.

An aspect of the present invention is, for example, the use of the compounds mentioned as pharmaceutical active compounds. Use as a pharmaceutical active compound relates to use for surgical, therapeutic or diagnostic procedures.

An aspect of the present invention relates to a pharmaceutical composition that comprises the compounds according to the present invention, if appropriate together with a pharmaceutically tolerable carrier.

In an embodiment of the present invention, pharmaceutical carriers can, for example, be fillers, extenders, binders, humectants, solution retardants, disintegrants, absorption accelerators, wetting agents, absorbents and/or glidants.

In an embodiment of the present invention, the compounds can be used as a ligand for a domain selected from the group comprising Src-homology-3 domains, WW domains, Ena-VASP-homology-1 domains, GYF domains, UEV domains and/or profilin.

In an embodiment of the present invention, all these domains interact with proline-rich sequences with affinities between 1 and 500 μM and in certain embodiments of the present invention optionally need further flanking epitopes to achieve the necessary specificity. The bonds between ligands, peptide and domain can come about, for example, by the interaction of two preformed hydrophobic surfaces. On the surface of the domain proteins, there can, for example, be an accumulation of aromatic amino acids, the radicals of which form a hydrophobic binding pocket. The proline-rich peptide ligand has a geometrically fixed, complementary structure owing to the rigid nature of the PPII helix, which comes into contact with the domain surface. There are here not hydrophobic contacts over the whole length of the core motif, instead, the ligand peptide can, for example, form an umbrella-like structure which spans the domain. Some of the proline radicals are accommodated in the hydrophobic binding pockets and thus interact with the aromatic radicals of the domain. If these contacts are not sufficient for a biologically relevant binding strength, additionally stabilizing hydrogen bridges can, for example, be formed, which is made possible by the electron-rich carbonyl group of the proline.

The intermolecular binding is favored by the restricted flexibility of the PPII helix, as owing to the relatively high degree of order, the entropy decrease during the bond formation is lower than in a customary linear peptide. For the quantification of this energy contribution, a dipeptide xP can be considered that has only two of the otherwise customary four degrees of rotational freedom around the peptide backbone. As each degree of rotational freedom corresponds to approximately 3.5 kJ/mol at 300 K, an energy advantage of about 7 kJ/mol per xP dipeptide results during the complex formation.

A further affinity increase is also observed by the multiple repetition of the proline-rich sequences in a sole peptide, such as, for example, the bacterial surface protein ActA, which binds to the EVH1 domain (Ena-VASP-homology-1 domain).

The EVH1 domain consists of approximately 115 amino acids and occurs in a multiplicity of signaling multi-domain proteins. In addition to some others, the family of the Ena/VASP proteins also belongs thereto, which act as molecular adapters and modulate the actin dynamics of the cytoskeleton. The EVH1 domains are subdivided into three classes according to their ligand preference. The first class in particular specifically recognizes an FPPPP core motif, which is found in all focal adhesion proteins such as vinculin and zyxin as well as in the ActA protein of the intracellular bacterium Listeria monocytogenes.

For the investigation of the processes at a molecular level and the resulting biological functioning, the three-dimensional domain structure was elucidated, but the interaction with various ligand peptides was also investigated. Without wishing to be limited to a particular theory, it is believed that the EVH1 domains recognize a consensus core motif FPxφP, in which phenylalanine (F) and the two outer proline positions (P) are needed for bond formation, but the two inner positions certainly permit variations (x=any desired amino acid, φ=hydrophobic amino acid). This has been investigated jointly in the class I EVH1 domain of the human VASP protein using a short section of the ActA peptide (₃₃₂SFEFPPPPTEDEL₃₄₄). The central FPPPP motif and an affinity-increasing EL epitope are framed and show a marked influence of the binding strength on substitution of the individual positions by other natural amino acids (dark=good binding, light=no binding).

The fact that the positions P₀ and P₁ can be replaced by other amino acids, P₀ especially being completely non-specific, can be explained by the consideration of the ligand peptide in the binding mode: while P₋₁ and P₂ are accommodated in a hydrophobic binding pocket of the domain, the central two prolines are situated in an umbrella-like position above the domain and have almost no contact with the domain surface. The carbonyl group of P₀, however, forms an hydrogen bridge to the NH of the tryptophan radical W23 of the EVH1 domain.

It was furthermore possible to obtain an overview of the binding affinities with the aid of a series of different test ligand peptides. The ligand ₃₃₂SFEFPPPPTEDEL₃₄₄ (third of the four proline-rich repeats of the ActA protein) yields the highest observed binding affinity (K_{D}=45 μM). On shortening the ligand to the core motif FPPPPT, however, measurable affinity for the VASP-EVH1 domain could no longer be determined, binding to the Mena-EVH1 domain, on the other hand, was very weak, but still detectable (417 μM).

In an embodiment of the present invention, the compounds can, for example, be employed as poly-proline mimetics. Proline-rich amino acid sequences can, for example, be found in peptides that are involved in signal transduction processes, such as in intracellular signal transduction processes. Within the meaning of the present invention, the term mimetics can also be understood as meaning analogs. The compounds according to the present invention can, for example, be used for the treatment of diseases that are associated with a modification of intracellular signal transduction processes, which are mediated by polyproline helix structures, selected from the group comprising bacterial infectious diseases, neurodegenerative diseases and/or tumors.

In an embodiment of the present invention, the bacterial diseases can, for example, be diseases that are associated, in particular mediated, by the following bacteria: Legionella, streptococci, staphylococci, Klebsiella, Hemophilis influenzae, Rickettsia (petechiae), mycobacteria, mycoplasmas, ureaplasmas, Neisseria (meningitis, Waterhouse-Friedrichsen syndrome, gonorrhoea), pseudomonads, Bordetella (pertussis), Corynebacteria (diphtheria), Chlamydia, Campylobacter (diarrhoea), Escherichia coli, Proteus, Salmonella, Shigella, Yersinia, vibrios, Enterococci, Clostridia, Borrelia, Treponema pallidum, Brucellae, Francisellae and/or Leptospira, in particular Listeria.

In an embodiment of the present invention, diseases include those that are caused by Listeria selected from the group comprising L. monocytogenes Sv1/2a, L. monocytogenes Sv4b F2365, L. monocytogenes Sv4b H7858, 178 contigs, L. monocytogenes Sv1/2a F6854, 133 contigs, L. monocytogenes Sv4b, L. monocytogenes Sv4a, L. innocua Sv6a, L. welshimeri Sv6b, L. seeligeri Sv1/2b and/or L. ivanovii Sv5 or are traced back to the Listeria mentioned.

In an embodiment of the present invention, neurodegenerative diseases can, for example, be selected from the group comprising Alzheimer's disease, Parkinson's disease, Huntington's disease and/or amyotrophic lateral sclerosis (ALS).

In an embodiment of the present invention, tumors can, for example, be selected from the group comprising tumors of the ear, nose and throat region, of the lung, of the mediastinum, of the gastrointestinal tract, of the urogenital system, of the gynecological system, of the breast, of the endocrine system, of the skin, bone and soft tissue sarcomas, mesotheliomas, melanomas, neoplasms of the central nervous system, cancers or tumors in childhood, lymphomas, leukemias, paraneoplastic syndromes, metastases without known primary tumor (CUP syndrome), peritoneal carcinomatoses, immunosuppression-related malignancies and/or tumor metastases.

The tumors can, for example, be the following types of cancer: adenocarcinoma of the breast, of the prostate and of the colon; all forms of lung cancer that starts from the bronchi; bone marrow cancer; melanoma; hepatoma; neuroblastoma; papilloma; apudoma; choristoma; branchioma; malignant carcinoid syndrome; carcinoid heart disease; carcinoma (for example Walker carcinoma, basal cell carcinoma, basosquamous carcinoma, Brown-Pearce carcinoma, ductal carcinoma, Ehrlich tumor, in-situ carcinoma, Krebs-2 carcinoma, Merkel cell carcinoma, mucous cancer, non-small cell bronchial carcinoma, oat-cell carcinoma, papillary carcinoma, scirrhous carcinoma, bronchioloalveolar carcinoma, bronchial carcinoma, squamous cell carcinoma and transitional cell carcinoma); histiocytic functional disorder; leukemia (for example in connection with B-cell leukemia, mixed cell leukemia, null cell leukemia, T-cell leukemia, chronic T-cell leukemia, HTLV-II-associated leukemia, acute lymphocytic leukemia, chronic lymphocytic leukemia, mast cell leukemia and myeloid leukemia); malignant histiocytosis, Hodgkin's disease, non-Hodgkin lymphoma, solitary plasma cell tumor; reticuloendotheliosis, chondroblastoma; chondroma, chondrosarcoma; fibroma; fibrosarcoma; giant-cell tumors; histiocytoma; lipoma; liposarcoma; leukosarcoma; mesothelioma; myxoma; myxosarcoma; osteoma; osteosarcoma; Ewing's sarcoma; synovioma; adenofibroma; adenolymphoma; carcinosarcoma; chordoma; craniopharyngioma; dysgerminoma; hamartoma; mesenchymoma; mesonephroma; myosarcoma; ameloblastoma; cementoma; odontoma; teratoma; thymoma; chorioblastoma; adenocarcinoma; adenoma; cholangioma; cholesteatoma; cylindroma; cystadenocarcinoma; cystadenoma; granulosa cell tumor; gynadroblastoma; hidradenoma; islet cell tumor; Leydig-cell tumor; papilloma; Sertoli-cell tumor; theca cell tumor; leiomyoma; leiomyosarcoma; myoblastoma; myoma; myosarcoma; rhabdomyoma; rhabdomyosarcoma; ependynoma; ganglioneuroma; glioma; medulloblastoma; meningioma; neurilemmoma; neuroblastoma; neuroepithelioma; neurofibroma; neuroma; paraganglioma; non-chromaffinic paraganglioma; angiokeratoma; angiolymphoid hyperplasia with eosinophilia; sclerosing angioma; angiomatosis; glomangioma; hemangioendothelioma; hemangioma; hemangiopericytoma, hemangiosarcoma; lymphangioma, lymphangiomyoma, lymphangiosarcoma; pinealoma; cystosarcoma phyllodes; hemangiosarcoma; lymphangiosarcoma; myxosarcoma; ovarian carcinoma; sarcoma (for example Ewing's sarcoma, experimental, Kaposi's sarcoma and mast cell sarcoma); neoplasms (for example bone neoplasms, breast neoplasms, neoplasms of the digestive system, colorectal neoplasms, hepatic neoplasms, pancreatic neoplasms, hypophyseal neoplasms, testicular neoplasms, orbital neoplasms, neoplasms of the head and neck, of the central nervous system, neoplasms of the hearing organ, of the pelvis, of the respiratory tract and of the urogenital tract); neurofibromatosis and cervical squamous dysplasia.

In an embodiment of the present invention, the cancer or the tumor can, for example, be selected from the group consisting of: tumors of the ear, nose and throat region comprising tumors of the inner nose, of the paranasal sinuses, of the nasopharynx, of the lips, of the oral cavity, of the oropharynx, of the larynx, of the hypopharynx, of the ear, of the salivary glands and paraganglioma, tumors of the lung comprising non-small cell bronchial carcinomas, small cell bronchial carcinomas, tumors of the mediastinum, tumors of the gastrointestinal tract comprising tumors of the esophagus, of the stomach, of the pancreas, of the liver, of the gallbladder and of the bile ducts, of the small intestine, colon and rectal carcinomas and anal carcinomas, urogenital tumors comprising tumors of the kidneys, of the ureter, of the bladder, of the prostate, of the urethra, of the penis and of the gonads, gynecological tumors comprising tumors of the cervix, of the vagina, of the vulva, uterine carcinoma, malignant trophoblastic disease, ovarian carcinoma, tumors of the oviduct (Fallopian tube), tumors of the abdominal cavity, mammary carcinomas, tumors of endocrine organs comprising tumors of the thyroid, of the parathyroid, of the adrenal cortex, endocrine pancreatic tumors, carcinoid tumors and carcinoid syndrome, multiple endocrine neoplasias, bone and soft tissue sarcomas, mesotheliomas, skin tumors, melanomas comprising cutaneous and intraocular melanomas, tumors of the central nervous system, tumors in childhood comprising retinoblastoma, Wilms' tumor, neurofibromatosis, neuroblastoma, Ewing's sarcoma tumor family, rhabdomyosarcoma, lymphomas comprising non-Hodgkin lymphoma, cutaneous T-cell lymphomas, primary lymphomas of the central nervous system, Hodgkin's disease, leukemias comprising acute leukemias, chronic myeloid and lymphatic leukemias, plasma cell neoplasms, myelodysplastic syndromes, paraneoplastic syndromes, metastases without known primary tumor (CUP syndrome), peritoneal carcinomatosis, immunosuppression-related malignancy comprising AIDS-related malignancies such as Kaposi's sarcoma, AIDS-associated lymphomas, AIDS-associated lymphomas of the central nervous system, AIDS-associated Hodgkin's disease and AIDS-associated anogenital tumors, transplantation-related malignancies, metastasized tumors comprising cerebral metastases, pulmonary metastases, hepatic metastases, bone metastases, pleural and pericardial metastases and malignant ascites.

In an embodiment of the present invention, the cancer or the tumor can, for example, be selected from the group comprising cancers or tumors of the breast, of the gastrointestinal tract, including colon carcinomas, gastric carcinomas, pancreatic carcinomas, colon cancer, small intestinal cancer, ovarian carcinomas, cervical carcinomas, lung cancer, prostate cancer, renal cell carcinomas and/or hepatic metastases.

In the treatment of the diseases mentioned, the pharmaceutical composition which comprises the compounds according to the present invention can, for example, be prepared and/or used as a gel, powder, powders, tablet, delayed release tablet, premix, emulsion, infusion formulation, drops, concentrate, granules, syrup, pellet, boli, capsule, aerosol, spray and/or inhalate.

In an embodiment of the present invention, the pharmaceutical composition which comprises the compounds according to the present invention can, for example, be present in a concentration of 0.1 to 99.5, for example, of 0.5 to 95.0, or, for example, of 1.0 to 90.0, or for example, of 10.0 to 85.0, or, for example, of 20.0 to 80.0%, by weight in a preparation.

In an embodiment of the present invention, this preparation can, for example, be employed orally, subcutaneously, intravenously, intramuscularly, intraperitoneally and/or topically.

In an embodiment of the present invention, the pharmaceutical composition which contains the compounds according to the present invention can, for example, be employed in total amounts of 0.05 to 500 mg per kg, for example, of 5 to 100 mg per kg of body weight, every 24 hours.

In an embodiment of the present invention, the bringing into contact with a body can, for example, be carried out orally, via injection, topically, vaginally, rectally and/or nasally.

In an embodiment of the present invention, the present invention also provides a kit which comprises at least one of the compounds according to the present invention and/or one of the pharmaceutical compositions according to the present invention, if appropriate, with information for combining the contents of the kit, for example, a package insert or an Internet address that refers to homepages with further information etc. The information for handling the kit can comprise, for example, a treatment scheme for the abovementioned diseases, in particular for the diseases set forth. The information can also, however, comprise information about how the products according to the present invention are to be employed within a diagnosis of the diseases mentioned. The kit according to the present invention can also be used in basic research.

The present invention accordingly also relates to the use of the kit for the prophylaxis and/or treatment of neurodegenerative diseases, bacterial infectious diseases or tumors.

The present invention is intended to be illustrated in more detail below with the aid of the synthesis of the diproline mimetic 85, without being restricted thereto.

Molecules were searched which should be able to bind to PRM-binding domains with high affinity and thus to replace the native proline-rich sequences of the ligand peptides as a binding partner. On the basis of the study of the interactions of ligand peptides in PRM-recognizing protein domains such as representatives of the WW domains, EVH1 domains, GYF domains and SH3 domains, compound 85 was designed, which could be incorporated into test peptides as a promising module (dipeptide mimetic) to replace two adjacent amino acid positions in the conformation of a left-handed polyproline II helix (PPII). Guidelines of the structural design were (1) a geometrically fixed structure that can replace an xP motif with x: any desired natural or unnatural amino acid, (2) a greatest possible agreement of the bonding angles and distances in comparison to those of xP dipeptides (in the PPII helix conformation), (3) a central hydrogen bridge acceptor function in the form of a carbonyl group, and (4) an amino acid-like overall structure with an Fmoc-protected N-terminus and a free C-terminus to make the planned incorporation into peptides methodically simple. These requirements are fulfilled by the molecules 85 and 86 depicted below, which are conformationally restricted analogs of a proline leucine unit in the PPII helix. By means of the incorporation of the Z-configured olefin bridge, the proline rings are stabilized in the presumed biologically active conformation; the central seven-membered ring providing a fixation of the trans-amide bond. The methyl group on the chiral center newly introduced by the bridging fills the binding pocket in both cases in the computer model.

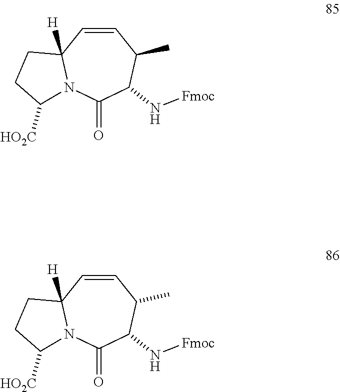

For the preparation of this compound in substantial amounts, a practicable, stereoselective synthesis route had to be worked out. For this purpose, a strategy was chosen in which the target molecule is led back in a convergent manner (retrosynthetic dissection of the central seven-membered ring, see Scheme 20) to the vinylprolines of the type 100 and the dehydroisoleucine 101. These could first of all be linked to one another by a peptide coupling before the tricycle was closed by olefin metathesis. The two differently substituted amino acid derivatives 100 and 101 in turn had to in each case be synthesized stereoselectively. As a starting material, proline was able to serve in one case and glycine in the second case.

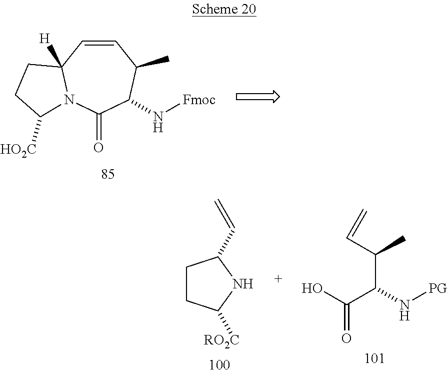

As a component of the type 101, the dehydroleucine 166 having an N-Boc protective group was proposed, the synthesis of which began with the conversion of glycine 133 to the ester 136 (Scheme 31).

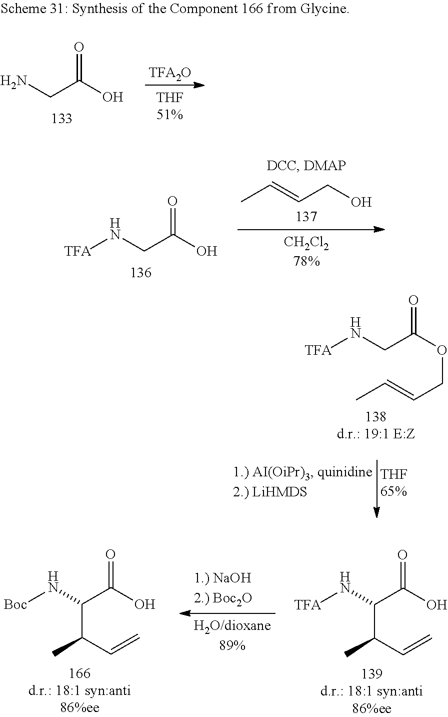

Glycine 133 was protected at the N-terminus in 51% yield using TFA₂O in THF. The TFA-protected amino acid 136 could then be esterified with E-crotyl alcohol 137 and yielded the allylglycine 138 in 78% yield. The Claisen rearrangement subsequently carried out according to the methodology of Kazmaier (U. Kazmaier, A. Krebs, Angewandte Chemie int. Ed., 1995, 34, 2012-2014) could be carried out successfully in a yield of 65% and with 86% ee (Lit.: 70%, 86% ee). By subsequent protective group manipulation, the Boc-protected component 166 was obtained with a yield of 89%.

For the synthesis of the second component, a cis-5-vinylproline ester of the type 100, recourse was made to a synthesis developed in our laboratories (Scheme 45).

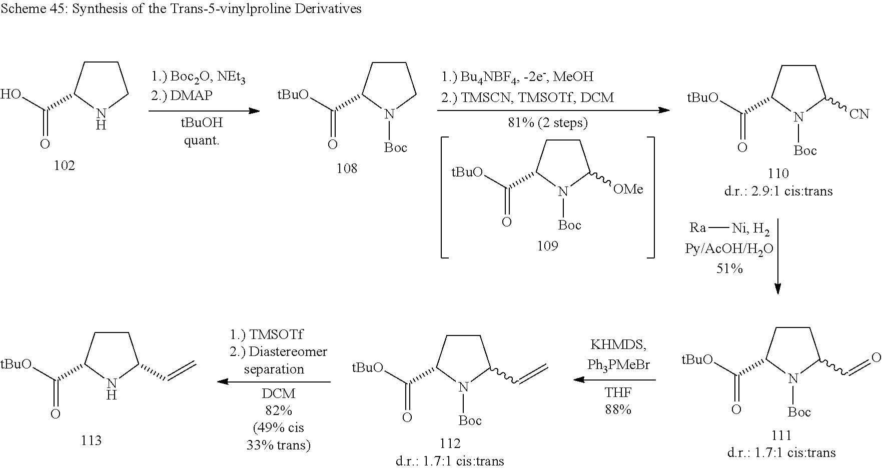

First, 50 g of L-proline 102 were protected at the C- and N-terminus in quantitative yield using Boc₂O, triethylamine and DMAP. The electrochemical oxidation of the protected proline 108 could be carried out on a 46 g (170 mmol) scale with an electrode surface of 48×56 mm and 240 mA constant current strength. The purification of the oxidation product led to high yield losses, which is why this was directly reacted further and only purified after the cyanation to give the nitrile 110. This was carried out in a 1 vol % solution of TMSOTf in DCM by dropwise addition of 1.1 eq of TMSCN and yielded over two stages a cis/trans isomer mixture of the cyanide 110 with a yield of 81% (d.r.: 2.9:1, cis:trans). The subsequent hydrogenation was carried out on the 18 g scale at 80° C. under a hydrogen atmosphere with Raney nickel in a mixture of pyridine, acetic acid and water (2:1:1) and yielded the aldehyde 111 in 51% yield. The use of a hydrogen atmosphere instead of NaH₂PO₂ represents a clear improvement in the known methodology. A Wittig reaction using KHMDS as a base yielded the desired product 112 in 88% yield obtained (d.r.: 1.7:1, cis:trans). Deprotection with TMSOTf in DCM and subsequent column-chromatographic diastereomer separation yielded the required cis-configured proline 113 in 49%, and the trans-epimer (not shown) in 33% yield.

Using the material thus obtained, the further synthesis of the tricyclic diproline mimetics was explored (peptide coupling and subsequent ring closure metathesis). The linkage of the peptide bond using PyBOP in MeCN at room temperature succeeded with a good yield of 75%. Here, the carboxylic acid 166 was preactivated with PyBOP before the addition of the amine 113 and diisopropylethylamine as a base took place. The identity of the coupling product could be confirmed in the ¹H-NMR spectrum despite the presence of a diastereomer mixture.

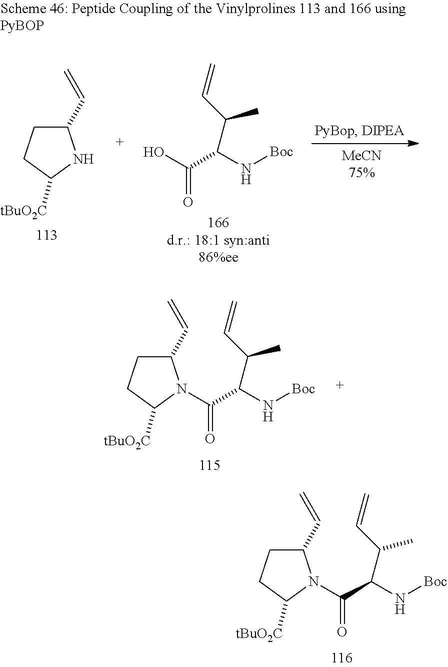

To produce the tricycles 117 and 118 by ring closure metathesis, the diastereomer mixture of the dipeptides 115 and 116 (Scheme 53) was treated with the Grubbs II catalyst (10 mol %), and refluxed for 24 hours in dichloromethane.

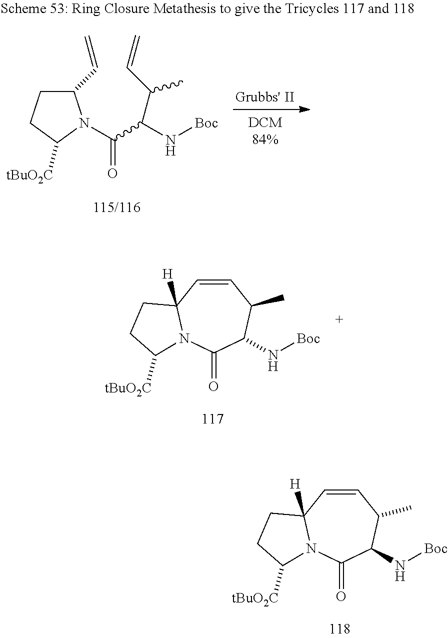 [SMILES: C=CC(C)C(NC(=O)OC(C)(C)C)C(=O)N1[C@@H](C=C)CC[C@H]1C(=O)OC(C)(C)C ; C[C@@H]1C=C[C@H]2CC[C@@H](C(=O)OC(C)(C)C)N2C(=O)[C@H]1NC(=O)OC(C)(C)C ; C[C@H]1C=C[C@H]2CC[C@@H](C(=O)OC(C)(C)C)N2C(=O)[C@@H]1NC(=O)OC(C)(C)C]

The desired product 117 was obtained in 83% yield. Moreover, 1% of the diastereomer 118 could be separated by column chromatography. The success of the ring closure metathesis could be confirmed unequivocally here in the ¹H-NMR spectrum with the aid of the change in the olefin signals. In addition, the structure and configuration of the tricycle 117 could be assigned by means of X-ray diffractometry.

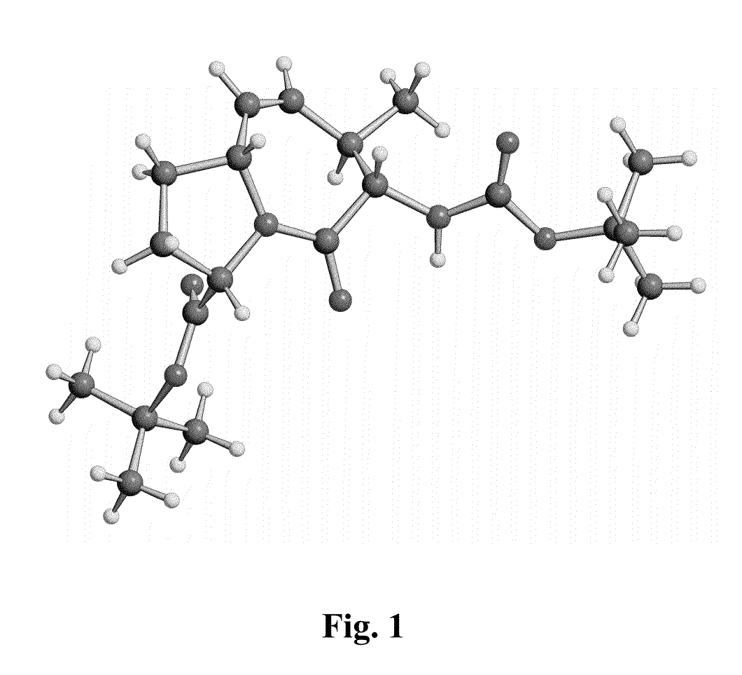

To make the synthesized bicycle 117 (as is shown in FIG. 1) usable for the solid-phase peptide synthesis, both protective groups had to be cleaved and the amine function had to be provided with an Fmoc protective group. This is the only N-protective group of practical importance that can be cleaved under mild basic conditions, for which generally secondary amines such as piperidine, morpholine, inter alia, are used. The generation of the unprotected amino function in the solid-phase synthesis is thereby made possible without additional production of salts. The increased base lability adversely affects the use of the Fmoc group in the organic synthesis and therefore necessitates the late change of the protective group.

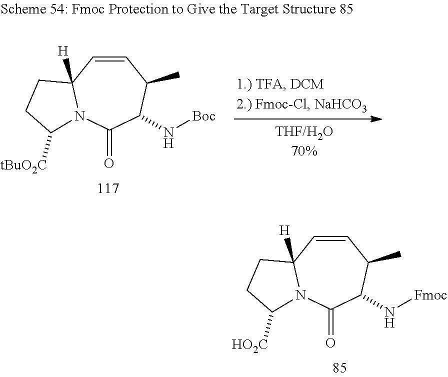

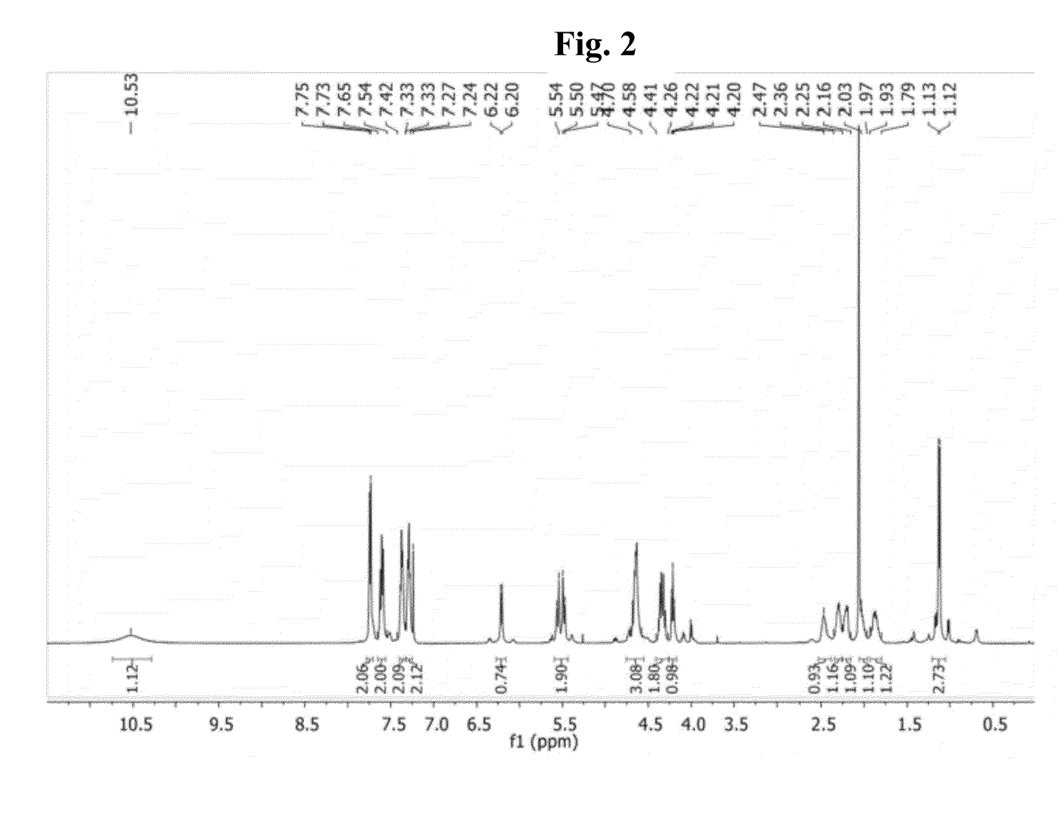

For the mutual acidic cleavage of the Boc protective group and of the tert-butyl ester, 117 was treated with an excess of trifluoroacetic acid (99% strength) in dichloromethane and stirred at room temperature (Scheme 54). The residue was subsequently converted under Schotten-Baumann conditions into the Fmoc-protected final product 85. This proved to be decidedly polar, but could nevertheless be extracted from the aqueous phase at pH≈1 with dichloromethane and subsequently purified by column chromatography on silica gel. 85 was obtained as an amorphous off-white solid (85%), the NMR spectra of which corresponded with the expectations (see FIG. 2 for 500 MHz ¹H-NMR spectrum (MeOH-d⁴), 60:40 rotamer mixture).

The use of the synthesis shown here, using the anti component 140 after peptide coupling, metathesis and protective group manipulation (cf. Schemes 46, 53 and 54), leads in an analogous manner to the bicycle 86.

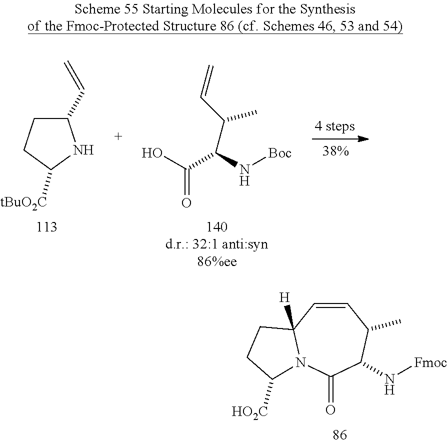

The anti component 140 required for this was prepared in 5 steps according to Scheme 56.

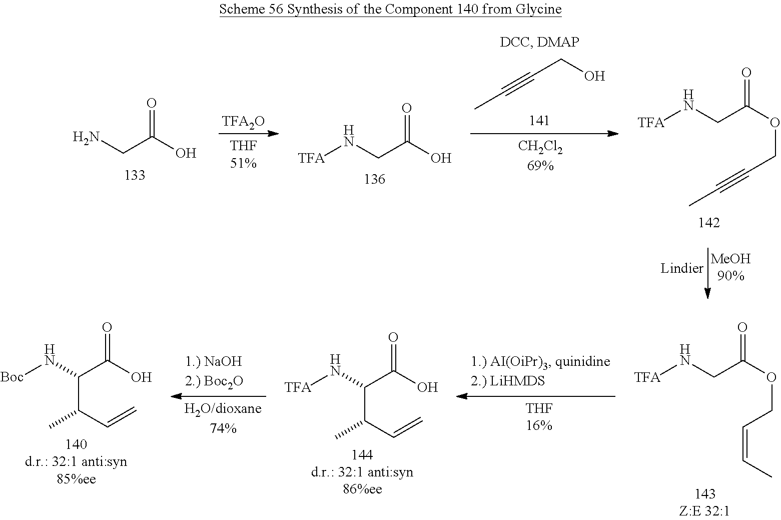

Glycine 133 was protected at the N-terminus in 51% yield using TFA₂O in THF. The TFA-protected amino acid 136 could then be esterified with the butyne alcohol 141 and yielded the ester 142 in 69% yield. After a Lindlar reduction, the Z-configured ester 143 was obtained with good selectivities (Z:E 32:1). The Claisen rearrangement subsequently carried out according to the methodology of Kazmaier (U. Kazmaier, A. Krebs, Angewandte Chemie int. Ed., 1995, 34, 2012-2014) could be carried out in a yield of 16% and with 86ee (Lit.: 70%, 86% ee). By subsequent protective group manipulation, the Boc-protected component 140 was obtained with a yield of 74%.

### Biological Results

Altogether, approximately 300 mg of the conformationally fixed diproline 85 were able to be synthesized and handed over to the FMP in Berlin-Buch for the purpose of incorporation into test ligand peptides and biological binding studies.
1) The incorporation of the synthetically prepared tricycle 85 into various test peptides under the standard coupling conditions (DIC/HOBt) of the solid-phase peptide synthesis proceeded without problems.
2) The predictions that the middle two proline positions of the F/WPPPP core motif should be replacable were confirmed.
3) The prediction of the reactive conformation on binding the PPII helix to the EVH1 receptor was confirmed by the good binding affinity of the ligand peptide I, or that the biologically required conformation and the actual conformation of the tricycle 85 agree.
4) The expected favorable entropy effect owing to local, conformational pre-fixing of the ligand appears confirmed by the higher binding affinity observed in comparison to the native peptide sequence. On the other hand, the geometrical fixing, however, also does not prevent the approach of ligand and domain protein and thus the bond formation.

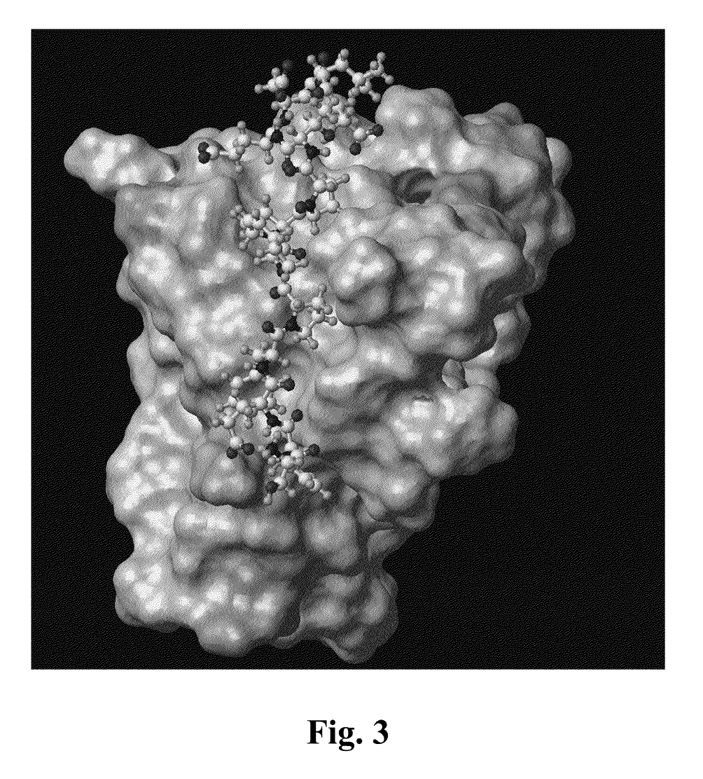

The VASP-EVH1 domain with ₃₃₂SFEFPₚₚPTEDEL₃₄₄peptide ligand (molecular modeling depiction) is shown in FIG. 3. It is easy to see the EVH1 domain as a “trench” on the surface of the VASP protein, which accommodates the ligand peptide.

### First Biological Results and Outlook

With the aid of the synthetic compound 85, ligand peptides with partially or completely replaced proline sequences were synthesized by way of example and investigated for their interaction with the EVH1 domain (pp=component 85). It was seen here by the incorporation of the mimetic 85 that the binding affinity of the ligand peptides to the domain could be increased. The replacement of all prolines by two components 85 linked to one another led to ligand peptides with an affinity comparable to that of the native peptide. This is summarized in Table 1 below, which lists the binding affinities of the ligand peptides and of the underlying native peptide ligands. The binding affinities were determined by means of NMR titration, the kinetic Biacore method or by means of fluorescence titration. The results are summarized in Table 1.

TABLE 1
Comparison of the measured binding affinities (interaction of the
ligand peptides with the VASP-EVH1 domain in solution).

| | Binding | | | |
|---|---|---|---|---|
| | affinity | Binding affinity | | Fluorescence |
| Ligand peptide | (NMR) | (Biacore) | | titration |
| SFEFPPPPTEDEL | 45 μM | 55.1 | μM | 37 ± 10 μM |
| SFEFPppPTEDEL (I) | 20-35 μM | 29.3-33.3 | μM | 47 ± 14 μM |
| SFEFppPPTEDEL | — | — | | 82 ± 30 μM |
| SFEFPPppTEDEL | — | — | | 26 ± 5 μM |
| SFEFppppTEDEL | — | — | | 45 ± 15 μM |
| SFEWPPPPTEDEL | not | 13.7 | μM | — |
| | determinable | | | |
| SFEWPppPTEDEL (II) | not | 3.7-12.5 | μM | — |
| | determinable | | | |

The replacement of all prolines by 2 components 85 linked to one another led to ligand peptides with an affinity comparable to that of the native peptide. For the first time, ligand peptides have been developed without proline-rich sequences, which are bound by PRM-binding domains. Since the general concept could be implemented successfully with respect to the synthesis and the biological use of the target structure 85, on this promising basis, numerous other possibilities open up, with the aid of small synthetic molecules selectively to address relevant protein domains which bind polyproline-containing ligands. In addition to the variation of existing molecular structures, a widening of the concept to the synthesis of ligands for other domains (Mena-EVH1, WW, SH3 and the like), the replacement of other than the central two proline units of the FPPPP motif or the replacement of PPP or even PPPP motifs by suitable structures produced by organic synthesis are also conceivable.

### Experimental Section

All reactions with air- or water-sensitive components were carried out under absolute conditions. For this, the glass apparatuses used were heated with a Bunsen burner flame in an oil pump vacuum (final pressure of 0.1-0.5 mbar) and flooded with argon after cooling, a vacuum/argon double tap glass apparatus being used. Syringes and needles for the transfer of reagents and solvents were dried in an oven at 85° C. and flushed several times with argon before use. Solid reagents were filled into the reaction flask in an argon countercurrent. Solvents were removed in a rotary evaporator at a water bath temperature of 40° C. and a pressure of 10-1013 mbar. The further drying of the substances generally took place at room temperature in an oil pump vacuum (0.1-0.5 mbar). The chemicals used were obtained commercially from customary companies such as Merck, Sigma-Aldrich, Fluka, Acros, Lancaster and Strem and usually employed without further purification. The concentration of organometallic reagents was determined according to Paquette et al. by titration against menthol using phenanthroline as an indicator. All solvents that were used for extraction and purification processes were distilled before use. Anhydrous solvents were obtained by appropriate procedures.

The purification of the substances took place by flash column chromatography on silica gel 60 (0.040-0.063 mm) from Merck and was carried out under slight overpressure (0.3-0.5 bar). Analytical thin-layer chromatography (TLC) for reaction monitoring was carried out with silica gel plates from Merck (silica gel 60F₂₅₄). The evaluation of the chromatograms took place in UV light (X=254 nm) and by staining with a KMnO₄ solution (3 g of KMnO₄, 20 g of K₂CO₃, 5 ml of 5% strength NaOH, 300 ml of H₂O) and subsequent heating with a hot air dryer. Analytical high pressure liquid chromatography (HPLC) was carried out using a Knauer system (HPLC pump K-1001, DAD K-2700 Wellchrom, lamp K-2701 Wellchrom, solvent organizer K-1500), using a Merck-Hitachi system (L-4000 A UV detector, D-6200A intelligent pump, differential refractometer Ri 71), a Merck-Hitachi system (L-7250A intelligent pump, L-7455 UV detector) or an Agilent 1100 HPLC-MS system. Further details are found with the measurements listed.

¹H- and ¹³C-NMR spectra were recorded at room temperature on the equipment AC 250, DPX 300 and DRX 500 of Bruker. The chemical shifts are indicated relative to the residual proton content or to the resonance of the solvent as an internal standard. The assignment of the signals was carried out by the recording of suitable 2D experiments (DEPT, APT, H-H-COSY, HMQC, HMBC, NOE) and by comparison with analogous compounds. For the assessment of the NMR spectra, the program Mestre-C with the analytical functions contained there was used. The numbering of the molecules indicated for the assignment of the signals is largely identical with IUPAC, but can also differ therefrom in some cases for reasons of clarity. Signal multiplicities are indicated as follows: (s=singlet, d=doublet, t=triplet, q=quartet, quint=quintet, br=broad signal, m=multiplet). Because of the hindered rotation of the carbamate protective groups at room temperature, many compounds were observed as a rotamer mixture and are described as such (broad peaks or double set of signals in the ¹H and ¹³C spectrum).

IR spectra were recorded at room temperature on an FT-IR Paragon 1000 from Perkin-Elmer as the ATR (Attenuated Total Reflectance) on a ZnSe crystal, to which the samples were applied as a solution. The absorption bands are indicated in wave numbers (\tilde{v} [cm⁻¹]) and characterized according to their relative intensity as follows: vs (very strong), s (strong), m (medium), w (weak), br (broad signal).

Mass determinations were carried out on the apparatuses MAT Incos 50 Galaxy System (EI) and MAT 900 (ESI, HRMS) from Finnigan. The inlet method (direct inlet (DIP) or GC-MS), the type of ionization (EI or ESI) and the ionization energy in [eV] are in each case indicated in brackets. The signals recorded relate to the ratio m/e and are indicated in their intensity relative to the base peak (100%).

Optical rotations were measured on a Polarimeter 343 plus from Perkin Elmer. The concentration of [α] is indicated in the unit [g/100 ml], the measuring temperature, the wavelength (generally 589 nm, in some cases additionally 546, 405, 365 and 334 nm) and the solvent are indicated in each case.

Elemental analyses were carried out using an Elementar Vario EL, it being possible to classify the mass percentage composition according to the elements C, H and N. Melting points were determined with the aid of a B-545 from Büchi and are not corrected.

The naming of the compounds was carried out with the aid of the autonomous program of the electronic Beilstein database according to the guidelines of the IUPAC. Slight deviations from this nomenclature result due to dependence of the naming on substrates known from the literature or the greater consideration of functional groups as a unit (e.g., protective groups) and changed priorities resulting therefrom. The determination of the stereochemistry was carried out according to the rules of Cahn-Ingold-Prelog. (Lit: R. S. Cahn, C. K. Ingold, V. Prelog, Angew. Chemie 1966, 78, 413).

### Example 1

### (S)-Di-tert-butyl pyrrolidine-1,2-dicarboxylate (108)

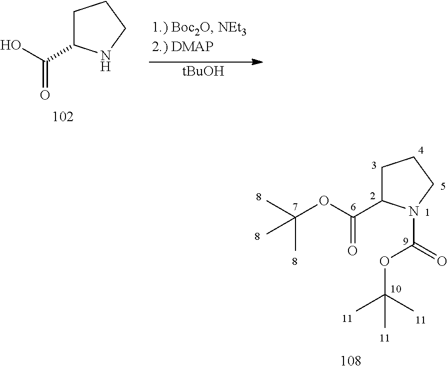 [SMILES: "tBuOH" ; O=C(O)[C@@H]1CCCN1 ; CC(C)(C)OC(=O)C1CCCN1C(=O)OC(C)(C)C]

52 ml (251 mmol, 2.7 eq) of Boc₂O were added to 10.1 g (87.73 mmol, 1.0 eq) of L-proline (102) in 180 ml of tBuOH and 3.70 ml (26.32 mmol, 0.3 eq) of NEt₃ and the solution was stirred for 4 h at room temperature until clear. 3.295 g (26.32 mmol, 0.3 eq) of DMAP were then added thereto and the solution was cooled in an ice bath until it began to solidify. It was stirred at room temperature for 17 h and the solution was heated to 45° C. for 1.5 h with vigorous stirring until evolution of gas was no longer observed. The batch was treated with 450 ml of MTBE, and washed successively with 300 ml each of 1N HCl soln., saturated NaHCO₃ soln. and saturated NaCl soln., the organic phase was dried over magnesium sulfate and the solvent was removed under reduced pressure. 23.8 g (87.70 mmol, 99%) of the protected proline 108 were obtained as a colorless oil. For analytical purposes, a small part of the product was purified by chromatography on silica gel with EtOAc/CyHex (1:9) as eluent.

M (C₁₄H₂₅NO₄): 271.35 g mol⁻¹.

TLC: R_{f}=0.12 (EtOAc/CyHex 1:9).

[α]²⁰_{D}: −53.1° (c=0.670, CHCl₃).

¹H-NMR (300 MHz, CDCl₃): δ=1.51-1.25 (m, 18H, H8, H11), 1.94-1.67 (m, 3H, H4, H3), 2.24-1.94 (m, 1H, H3′), 3.54-3.17 (m, 2H, H5), 4.10 (Ψ-ddd, J 25.5(Ψ), 8.6, 2.9, 1H, H2).

¹³C-NMR (75 MHz, CDCl₃): δ=24.05/23.26 (t, C4), 28.21/27.84 (q, C8, C11), 30.74/29.73 (t, C3), 46.35/46.15 (t, C5), 59.52 (d, C2), 79.40/79.20 (s, C10), 80.61 (s, C7), 154.14/153.79 (s, C9), 172.12 (s, C6).

GC-MS: m/e=271 ([M]⁺, 1), 170 ([M]⁺-C₅H₉O₂, 15), 142 (9), 114 (95), 57 (100), 19 (26).

IR (FT-ATR): ^{˜}v=2973 (s), 2928 (m), 2880 (w), 1738 (s), 1697 (s), 1476 (m), 1454 (m), 1396 (s), 1364 (s), 1289 (m), 1252 (s), 1215 (s), 1148 (s), 1085 (s), 1028 (w), 978 (m), 939 (m), 917 (m), 895 (m), 851 (m), 840 (m), 797 (w), 770 (m).

### Example 2

### (S)-Di-tert-butyl 5-methoxypyrrolidine-1,2-dicarboxylate (109)

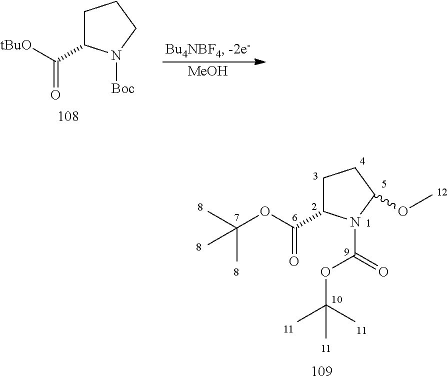 [SMILES: "MeOH" ; CC(C)(C)OC(=O)[C@@H]1CCCN1C(=O)OC(C)(C)C ; COC1CC[C@@H](C(=O)OC(C)(C)C)N1C(=O)OC(C)(C)C]

Two graphite plate electrodes (48×56 mm) were immersed at a distance of 5 cm in a solution of 46.129 g (170.00 mmol) of the protected proline 108 (0.5 M) and 5.6 g (17 mmol) of Bu₄NBF₄ (0.05 M) in 340 ml of methanol at 0° C. and electrolyzed at 240 mA constant current strength. After the passage of 2.46 F mol⁻¹, the solvent was removed under reduced pressure, and filtered through silica gel using 1000 ml of EtOAc/CyHex (1:1) as eluent. The solvent was removed under reduced pressure and 50.624 g (167.97 mmol, 99%) of crude product 109 were obtained as a clear oil and reacted further without further purification. For analytical purposes, a small part of the product was purified by chromatography on silica gel using EtOAc/CyHex (1:7) as eluent.

M (C₁₅H₂₇NO₅): 301.38 g mol⁻¹.

TLC: R_{f}=0.43 (EtOAc/CyHex 1:7).

¹H-NMR (300 MHz, CDCl₃): δ=1.59-1.23 (m, 18H, H8, H11), 2.45-1.62 (m, 4H, H3, H4), 3.63-3.20 (m, 3H, H12), 4.29-3.96 (m, 1H, H2), 5.34-4.99 (m, 1H, H5).

¹³C-NMR (75 MHz, CDCl₃): δ=28.23/28.15/27.92/27.82 (C8, C11), 30.82/29.79 (t, C3), 32.89/32.17 (t, C4), 32.89/32.17 (t, C4), 60.24/59.93/59.83/59.62 (d, C2), 80.90/80.69/80.36/80.27 (s, C7, C10), 89.22/88.34 (d, C5), 154.43/154.21/153.81 (s, C9), 171.73/171.67/171.59 (s, C6).

GC-MS: m/e=301 ([M]⁺, 1), 200 ([M]⁺-C₅H₉O₂, 22), 172 (8), 144 (32), 100 (80), 57 (100), 29 (14).

IR (FT-ATR): ^{˜}v=2975 (s), 2928 (s), 2825 (w), 1738 (s), 1699 (s), 1476 (m), 1455 (m), 1364 (s), 1327 (s), 1252 (s), 1157 (s), 1084 (s), 1024 (m), 987 (s), 939 (s), 911 (s), 885 (s), 841 (s), 797 (s), 772 (s), 729 (m).

### Example 3

### (S)-Di-tert-butyl 5-cyanopyrrolidine-1,2-dicarboxylate (110)

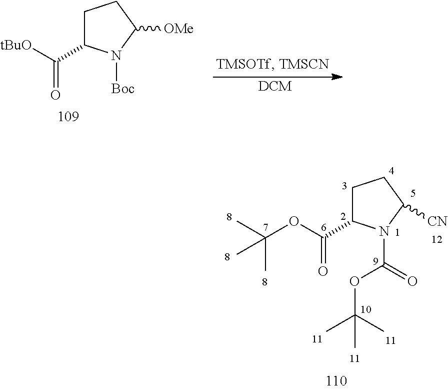

4.4 ml of TMSOTf (1 vol %), then 25.8 ml (191.81 mmol, 1.1 eq) of TMSCN were first added dropwise at −40° C. to 50.624 g (167.97 mmol, 1.0 eq) of the crude proline 109 in 440 ml of dry DCM and the mixture was stirred at this temperature for 1.5 h. The solution was treated with 65 ml of MeOH, the solvent was removed under reduced pressure and 40.569 g (136.89 mmol, 81% over two steps) (d.r.: 3:1 cis:trans) of a diastereomer mixture of the nitrile 110 were obtained by chromatography on neutral alumina using EtOAc/CyHex 1:9 as eluent. For analytical purposes, a small part of the individual isomers was separated by chromatography.

M (C₁₅H₂₄N₂O₄): 296.36 g mol⁻¹.

trans TLC: R_{f}=0.33 (EtOAc/CyHex 1:9).

trans [α]²⁰_{D}: −88.6° (c=0.550, CHCl₃).

trans melting point: 106.3° C.

trans ¹H-NMR (300 MHz, CDCl₃): δ=1.48-1.32 (m, 18H, H8, H11), 2.53-1.97 (m, 4H, H3, H4), 4.20 (Ψ-dd, J 26.2(Ψ), 7.9, 1H, H2), 4.61 (Ψ-dd, J 30.2(Ψ), 7.2, 1H, H5).

trans ¹³C-NMR (75 MHz, CDCl₃): δ=28.04/27.81 (q, C8, C11), 29.60/28.59/28.46 (t, C3, C4), 47.59/47.48 (d, C5), 59.52/59.41 (d, C2), 81.84/81.78/81.70/81.49 (s, C7, C10), 118.82/118.75 (s, C12), 152.77/152.48 (s, C9), 170.79/170.70 (s, C6).

trans GC-MS: m/e=255 ([M]⁺, 1), 199 (5), 154 ([M]⁺-C₅H₉O₂, 100).

trans IR (FT-ATR): ^{˜}v=2973 (m), 2928 (w), 2238 (w), 1734 (s), 1704 (s), 1473 (w), 1455, 1364 (s), 1323 (m), 1306 (m), 1252 (m), 1225 (s), 1144 (s), 1119 (s), 1061 (m), 1034 (w), 987 (w), 914 (m), 842 (m), 820 (w), 773 (m), 756 (w).

trans HR-MS: (ESI, C₁₅H₂₄N₂NaO₄): 319.163±0.001 u (319.1634 u).

cis TLC: R_{f}=0.27 (EtOAc/CyHex 1:9).

cis [α]²⁰_{D}: 19.2° (c=0.420, CHCl₃).

cis melting point: 81.3° C.

cis ¹H-NMR (300 MHz, CDCl₃): δ=1.63-1.33 (m, 18H, H8, H10), 2.47-2.00 (m, 4H, H3, H4), 4.16 (Ψ-td, J 14.2(Ψ), 6.7, 6.7, 1H, H2), 4.55 (Ψ-td, J 39.1(Ψ), 5.7, 5.7, 1H, H5).

cis ¹³C-NMR (75 MHz, CDCl₃): δ=28.14/27.84 (q, C8, C11), 30.39/29.71/29.64/28.57 (t, C3, C4), 47.52 (d, C5), 60.24/60.19 (d, C2), 82.01/81.91/81.62 (s, C7, C10), 118.38/118.09 (s, C12), 152.50/152.25 (s, C9), 170.53/170.30 (s, C6).

cis GC-MS: m/e=255 ([M]⁺, 1), 199 (5), 154 ([M]⁺-C₅H₉O₂, 100).

cis IR (FT-ATR): ^{˜}v=2976 (s), 2982 (m), 2880 (w), 2238 (w), 1738 (s), 1697 (s), 1473 (m), 1455 (m), 1384 (s), 1364 (s), 1286 (s), 1255 (s), 1215 (s), 1150 (s), 1116 (s), 1072 (s), 1028 (m), 986 (m), 950 (m), 935 (m), 905 (m), 878 (m), 842 (s), 769 (s).

cis HR-MS: (ESI, C₁₅H₂₄N₂NaO₄): 319.163±0.001 u (319.1634 u).

### Example 4

### (S)-Di-tert-butyl 5-formylpyrrolidine-1,2-dicarboxylate (111)

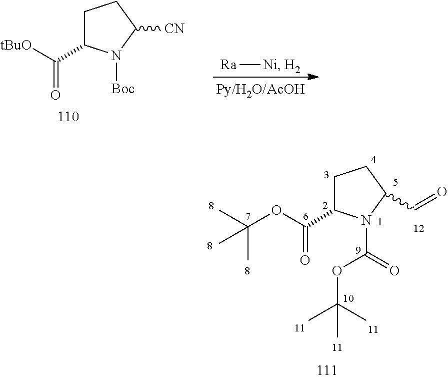

90 g of Raney Ni/H₂O (50 w % in water, Acros) were added to a solution of 17.905 g (60.42 mmol) of the cyanide 110 in 640 ml of Py/AcOH/H₂O (2:1:1), and the mixture was stirred under hydrogen for 72 h at 80° C. The suspension was treated with 300 ml of water and extracted three times with 800 ml each of MTBE. The combined organic phases were washed twice with water, dried over magnesium sulfate and the solvent was removed under reduced pressure. The residue was filtered through silica gel using 1000 ml of EtOAc/CyHex (1:1) and the crude product was purified by chromatography on silica gel using EtOAc/CyHex 1:4 as eluent after removal of the solvent. 9.234 g (30.85 mmol, 51%) of the aldehyde 111 were obtained as a yellow oil (d.r.: 1.7:1 cis:trans).

M (C₁₅H₂₅NO₅): 299.36 g mol⁻¹.

TLC: R_{f}=0.27 (EtOAc/CyHex 1:4).

¹H-NMR (300 MHz, CDCl₃): δ=1.50-1.34 (m, 18H, H8, H11), 2.25-1.83 (m, 4H, H3, H4), 4.53-3.84 (m, 2H, H2, H5), 9.65-9.45 (m, 1H, H12).

¹³C-NMR (75 MHz, CDCl₃): δ=29.94/29.20/29.00/28.27/26.29/26.17/25.24/24.56 (t, C3, C4), 28.14/27.90 (q, C8, C11), 60.52/60.38/60.29 (d, C5), 65.28 (d, C2), 81.63/81.54/81.45/81.35/81.23/81.00/80.77 (s, C7, C10), 154.12/153.93/153.55/153.15 (s, C9), 171.54/171.42/171.26 (s, C6), 200.99/200.11 (s, C12).

GC-MS: m/e=299 ([M]⁺, 1), 270 ([M]⁺-CO, 5), 198 ([M]⁺-C₅H₉O₂, 2), 170 (27), 142 (8), 114 (100), 98 (41), 57 (75), 39 (25).

IR (FT-ATR): ^{˜}v=2976 (s), 2926 (m), 2873 (w), 2806 (w), 2713 (w), 1730 (s), 1695 (s), 1476 (m), 1455 (m), 1383 (s), 1364 (s), 1290 (m), 1253 (s), 1220 (s), 1148 (s), 1123 (s), 1090 (s), 1010 (m), 979 (m), 913 (m), 843 (m), 796 (w), 771 (m).

### Example 5

### (S)-Di-tert-butyl 5-vinylpyrrolidine-1,2-dicarboxylate (112)

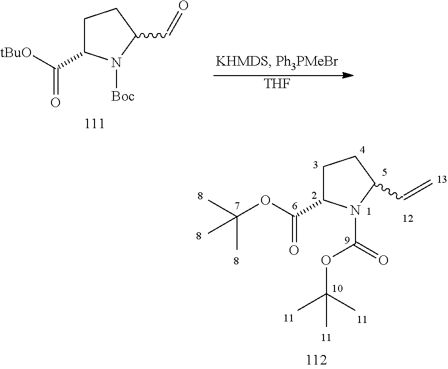 [SMILES: CC(C)(C)OC(=O)[C@@H]1CCC(C=O)N1C(=O)OC(C)(C)C ; C=CC1CC[C@@H](C(=O)OC(C)(C)C)N1C(=O)OC(C)(C)C ; "THF"]

93.5 ml (61.69 mmol, 2.0 eq) of a solution of KHMDS in toluene (15 w %, ABCR) were added under argon at room temperature to a suspension of 22.037 g (61.69 mmol, 2.0 eq) of Ph₃PMeBr in 220 ml of THF and the mixture was stirred for 1 h. A solution of 9.234 g (30.88 mmol, 1.0 eq) of the aldehyde 111 in 70 ml of THF was then added dropwise at −78° C. and stirred at room temperature for 2.5 h. The reaction mixture was treated with 160 ml of satd. Rochelle salt solution and 100 ml of water, extracted three times with 260 ml each of MTBE, and the combined organic phases were washed with satd. NaCl solution and dried over magnesium sulfate. The solvent was removed under reduced pressure and after column-chromatographic purification on silica gel using EtOAc/CyHex (1:9) as eluent 8.091 g (27.21 mmol, 88%) of the vinylproline 112 were obtained as an isomer mixture (d.r.: 1.7:1 cis:trans).

M (C₁₆H₂₇NO₄): 297.39 g mol⁻¹.

TLC: R_{f}=0.27 (EtOAc/CyHex 1:9).

¹H-NMR (400 MHz, CDCl₃): δ=1.50-1.32 (m, 18H, H8, H11), 2.19-1.59 (m, 4H, H3, H4), 4.58-4.01 (m, 2H, H2, H5), 5.44-4.92 (m, 2H, H13), 5.95-5.60 (m, 1H, H12).

¹³C-NMR (100 MHz, CDCl₃): δ=28.32/27.98 (q, C8, C11), 31.54/30.86/29.92/29.00/27.33 (t, C3, C4), 61.00/60.53/60.34/60.17/59.61/59.36 (d, C2, C5), 80.85/79.86/79.78/79.72 (s, C7, C10), 114.93/114.78/114.59/113.82/113.64 (t, C13), 139.21/138.97/138.63/138.50/138.06 (d, C12), 153.73/153.58 (s, C9), 172.09/172.03/171.97 (s, C6).

GC-MS: m/e=297 ([M]⁺, 1), 196 ([M]⁺-C₅H₉O₂, 168 (10), 140 (100), 114 (2), 96 (70), 79 (5), 57 (72), 39 (27).

IR (FT-ATR): ^{˜}v=2975 (s), 2933 (m), 2880 (w), 1737 (s), 1697 (s), 1640 (w), 1477 (m), 1455 (m), 1385 (s), 1363 (s), 1323 (m), 1290 (m), 1255 (m), 1213 (m), 1150 (s), 1106 (s), 1066 (w), 1023 (w), 988 (w), 957 (w), 912 (m), 873 (w), 856 (w), 843 (w), 770 (w).

HR-MS: (ESI, C₁₆H₂₇NNaO₄): 320.183±0.001 u (320.1838 u).

### Example 6

### (2S,5S)-tert-Butyl 5-vinylpyrrolidine-2-carboxylate (113)

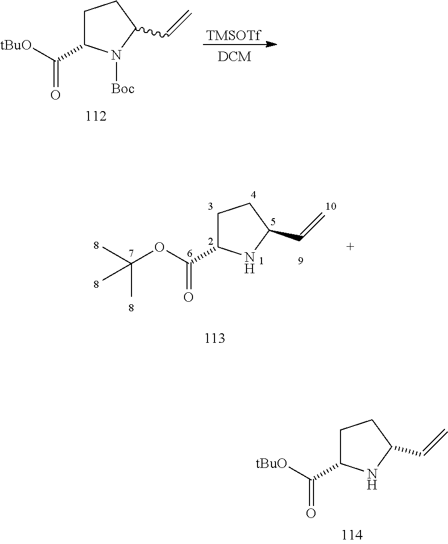 [SMILES: "TMSOTf" ; C=CC1CC[C@@H](C(=O)OC(C)(C)C)N1C(=O)OC(C)(C)C ; C=C[C@@H]1CC[C@@H](C(=O)OC(C)(C)C)N1 ; C=C[C@H]1CC[C@@H](C(=O)OC(C)(C)C)N1]

2.5 ml (13.82 mmol, 1.0 eq) of TMSOTf were added slowly to a solution of 4.041 g (13.59 mmol, 1.0 eq) of the vinylproline 112 in 50 ml of DCM under argon at 0° C., the mixture was stirred for 5 min and the reaction was ended by addition of 10 ml of saturated NaHCO₃ soln. The aqueous phase was extracted three times with 16 ml each of DCM, the combined organic phases were dried over magnesium sulfate and the solvent was removed under reduced pressure. After chromatography on silica gel using DCM/MeOH (25:1) as eluent, 887 mg (4.50 mmol, 33%) of the trans isomer 113 and 1.306 g (6.63 mmol, 49%) of the cis isomer 114 were obtained.

M (C₁₁H₁₉NO₂): 197.27 g mol⁻¹.

trans TLC: R_{f}=0.35 (DCM/MeOH 25:1).

trans [α]²⁰_{D}: −28.9° (c=0.715, CHCl₃).

trans ¹H-NMR (300 MHz, CDCl₃): δ=1.42 (s, 9H, H8), 1.58-1.45 (m, 1H, H4′), 1.96-1.70 (m, 2H, H3, H4), 2.25-2.10 (m, 1H, H3′), 2.70 (s, 1H, NH), 3.77-3.65 (m, 2H, H2, H5), 4.97 (d, J 10.2, 1H, H10), 5.13 (dd, J 17.1, 0.9, 1H, H10′), 5.76 (ddd, J 17.1, 10.1, 7.0, 1H, H9).

trans ¹³C-NMR (75 MHz, CDCl₃): δ=28.00 (q, C8), 29.60, 31.79 (t, C3, C4), 59.79, 60.89 (d, C2, C5), 81.01 (s, C7), 114.50 (t, C10), 140.89 (d, C9), 174.90 (s, C6).

trans GC-MS: m/e=197 ([M]+, 1), 96 ([M]+-C5H9O2, 100), 79 (12), 57 (22), 41 (32), 19 (10).

trans IR (FT-ATR): ^{˜}v=3346 (w), 3076 (w), 2974 (s), 2932 (m), 2872 (w), 1723 (s), 1641 (w), 1604 (w), 1591 (w), 1477 (m), 1456 (m) 1391 (s), 1366 (s), 1339 (m), 1226 (s), 1153 (s), 1029 (m), 991 (s), 916 (s), 846 (s), 808 (m), 754 (m) 721 (w), 692 (w), 677 (w).

trans HR-MS: (ESI, C₁₁H₂₀NO₂): 198.149±0.002 u (198.1494 u).

cis TLC: R_{f}=0.27 (DCM/MeOH 25:1).

cis [α]²⁰_{D}: −25.1° (c=1.475, CHCl₃).

cis ¹H-NMR (400 MHz, CDCl₃): δ=1.44 (s, 9H, H8), 2.28-1.82 (m, 4H, H3, H4), 2.22 (s, 1H, NH), 3.59 (dd, J 14.3, 7.2, 1H, H5), 3.67 (dd, J 8.7, 5.2, 1H, H2), 5.03 (d, J 10.3, 1H, H10), 5.17 (d, J 17.1, 1H, H10′) 5.85 (ddd, J 17.2, 10.2, 7.1, 1H, H9).

cis ¹³C-NMR (100 MHz, CDCl₃): δ=28.02 (q, C8), 31.87, 30.37 (t, C3, C4), 60.70 (d, C2), 62.23 (d, C5), 81.13 (s, C7), 115.20 (t, C10), 139.97 (d, C9), 174.33 (s, C6).

cis GC-MS: m/e=197 ([M]+, 1), 96 ([M]+-C5H9O2, 100), 79 (12), 57 (22), 41 (32), 19 (10).

cis IR (FT-ATR): ^{˜}v=3360 (w, br), 2977 (s), 2926 (m), 2873 (w), 1727 (s), 1636 (w), 1473 (w), 1453 (m), 1426 (w), 1390 (m), 1366 (s), 1283 (m), 1246 (s), 1225 (s), 1154 (s), 1100 (m), 1030 (w), 992 (m), 917 (m), 848 (s), 769 (w), 753 (w), 740 (w).

cis HR-MS: (ESI, C₁₁H₂₀NO₂): 198.149±0.001 u (198.1494 u).

### Example 7

### 2-(2,2,2-Trifluoroacetamido)acetic acid (136)

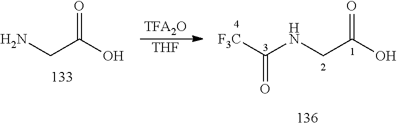 [SMILES: "TFA2O" ; NCC(=O)O ; O=C(O)CNC(=O)C(F)(F)F ; "THF"]

23 ml (160.46 mmol, 2.0 eq) of TFA₂O were added at 0° C. to a suspension of 6.023 g (80.23 mmol, 1.0 eq) of glycine 133 in 150 ml of THF and the mixture was subsequently stirred at room temperature for 3 h. The solvent was removed under reduced pressure, the residue was refluxed in 120 ml of CHCl₃/petroleum ether 1:1 and the product 136 was filtered off as a white solid. The filtrate was concentrated again and taken up in 40 ml of CHCl₃/petroleum ether 1:1, refluxed and further product 136 was filtered off as a white solid. 6.991 g (40.87 mmol, 51%) of the TFA-protected glycine 136 were obtained as a white powder.

M (C₄H₄F₃NO₃): 171.07 g mol⁻¹.

Mp.: 123.8° C.

¹H-NMR (300 MHz, CD₃OD): δ (ppm)=4.01 (s, 2H, H2).

¹³C-NMR (75 MHz, CD₃OD): δ (ppm)=41.69 (t, C2), 117.34 (q, J 286.1, C4), 159.47 (q, J 37.6, C3), 171.54 (s, C1).

IR (ATR) ¹⁷ (cm⁻¹)=3312 (s), 3102 (s), 2946 (m), 2570 (w), 2485 (w), 2438 (w), 1699 (s), 1557 (s), 1494 (m), 1407 (s), 1350 (s), 1154 (s, br), 1086 (s), 1014 (s), 964 (m), 942 (s), 845 (s), 772 (s), 730 (s), 681 (s).

### Example 8

### (E)-But-2-en-1-yl 2-(2,2,2-trifluoroacetamido)acetate (138)

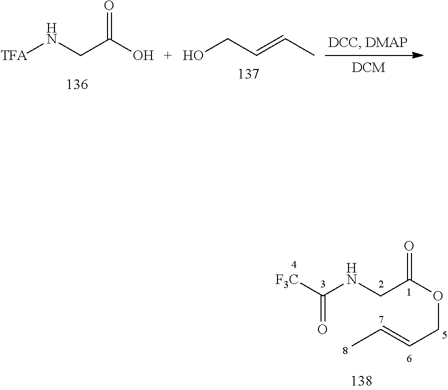

545 mg (4.46 mmol, 0.3 eq) of DMAP were added at 0° C. to a solution of 2.542 g (12.86 mmol, 1.0 eq) of TFA glycine 136, 3.373 g (16.35 mmol, 1.1 eq) of DCC and 1.39 ml (16.35 mmol, 1.1 eq) of E-crotyl alcohol 137 (Alfa-Aesar, d.r.: 19:1 E:Z) in 45 ml of DCM and the reaction mixture was stirred overnight at room temperature. The resulting urea was filtered off, the organic phase was washed successively with 45 ml each of water, 1 N HCl soln., and satd. NaCl soln., dried over magnesium sulfate and the solvent was removed at reduced pressure. After column-chromatographic purification on silica gel with EtOAc/CyHex 1:5 as eluent, 2.569 g (11.41 mmol, 78%) of the product 138 were obtained as a yellowish solid.

M (C₈H₁₀F₃NO₃): 225.17 g mol⁻¹.

TLC: R_{f}=0.30 (EtOAc/CyHex 1:5).

Melting point: 46.3° C.

¹H-NMR (300 MHz, CDCl₃): δ=1.63 (d, J 6.2, 3H, H8), 4.00 (s, 2H, H2), 4.50 (d, J 5.7, 2H, H5), 5.57-5.37 (m, 1H, H6), 5.86-5.61 (m, 1H, H7), 7.54 (s, 1H, NH).

¹³C-NMR (75 MHz, CDCl₃): δ=17.39 (q, C8), 41.05 (t, C2), 66.45 (t, C5), 115.53 (q, J 287.0, C4), 123.85 (d, C6), 132.54 (d, C7), 157.51 (q, J 37.8, C3), 168.01 (s, C1).

GC-MS: m/e=225 ([M]⁺, 1), 154 ([M]⁺-C₄H₇O, 10), 126 ([M]⁺-C₅H₇O₂, 50), 78 (20), 69 (30), 55 ([M]+-C₄H₃F₃NO₃, 100).

IR (FT-ATR): ^{˜}v=3258 (s), 3097 (w), 3023 (w), 2950 (w), 2889 (w), 1741 (s), 1716 (s), 1702 (s), 1672 (w), 1555 (s), 1454 (w), 1407 (m), 1389 (m), 1353 (m), 1263 (s), 1243 (w), 1163 (s), 1075 (w), 1010 (s), 969 (s), 934 (m), 887 (w), 846 (w), 781 (w), 711 (s), 684 (s).

### Example 9

### (2S,3R)-3-Methyl-2-(2,2,2-trifluoroacetamido)pent-4-enoic acid (139)

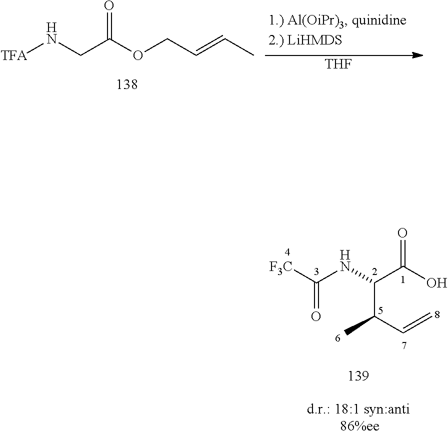

39 ml (38.95 mmol, 5.0 eq) of LiHMDS solution in THF (Aldrich, 1 M) were added dropwise at −78° C. over 35 min to a solution of 1.753 g (7.79 mmol, 1.0 eq) of the ester 138, 1.749 g (8.56 mmol, 1.1 eq) of Al(OiPr)₃ and 6.318 g (19.48 mmol, 2.5 eq) of quinidine in 130 ml of THF and the solution was slowly warmed to room temperature with stirring overnight. After addition of 400 ml of satd. NaHCO₃ soln. it was washed twice with 400 ml each of MTBE, the aqueous phase was brought to pH=1 in a separating funnel by addition of solid KHSO₄ and subsequently extracted three times with 400 ml each of DCM. The combined organic phases were dried over magnesium sulfate and the solvent was removed at reduced pressure. 1.145 g (5.09 mmol, 65%) of the rearranged product 139 were obtained as a yellow oil (86% ee, d.r.: 18:1 syn:anti).

M (C₈H₁₀F₃NO₃): 225.17 g mol⁻¹.

[α]²⁰_{D}: 83.8° (86% ee, d.r.: 18:1 syn:anti)(c=1.035, CHCl₃).

¹H-NMR (300 MHz, CDCl₃): δ=1.12 (d, J 7.0, 3H, H6), 2.87-2.71 (m, 1H, H5), 4.65 (dd, J 8.5, 4.7, 1H, H2), 5.18-5.04 (m, 2H, H8), 5.69 (ddd, J 17.7, 10.5, 7.6, 1H, H7), 6.95 (d, J 8.2, 1H, NH), 9.20 (s, 1H, COOH).

¹³C-NMR (75 MHz, CDCl₃): δ=15.00 (q, C6), 40.15 (d, C5), 56.04 (d, C2), 109.86/113.66/118.24/121.28 (s, C4), 117.53 (t, C8), 136.99 (d, C7), 157.78/157.28/156.78/156.28 (s, C3), 173.77 (s, C1).

GC-MS: m/e=229 ([M]⁺, 1), 173 (3), 156 ([M]⁺-C₄H₉O, 2), 128 ([M]⁺-C₅H₉O₂, 8), 112 (17), 84 (10), 74 (13), 57 (100).

IR (FT-ATR): ^{˜}v=3283 (m, br), 3087 (m, br), 2976 (m, br), 2624 (w, br), 1711 (s), 1650 (m), 1643 (m), 1555 (s), 1536 (s), 1454 (m), 1416 (m), 1384 (m), 1356 (m), 1278 (s), 1206 (s), 1162 (s, br), 1023 (m), 994 (s), 924 (s), 860 (w), 803 (m), 770 (m), 727 (s), 696 (s).

### Example 10

### (2S,3R)-2-((tert-Butoxycarbonyl)amino)-3-methylpent-4-enoic acid (166)

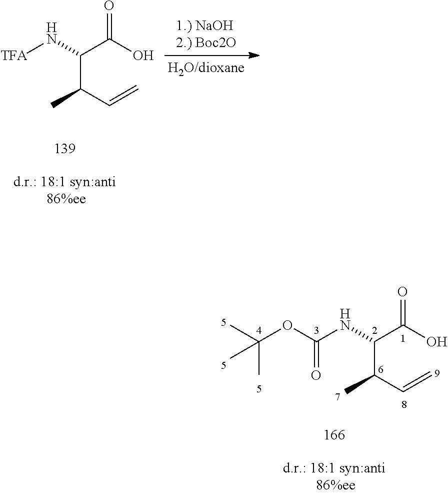

9 ml (8.94 mmol, 2.5 eq) of a 1 N aqueous NaOH soln. were added to a solution of 805 mg (3.58 mmol, 1.0 eq) of the TFA-protected acid 139 in 9 ml of dioxane and the solution was stirred at room temperature for 22 h. The pH was checked (pH≧8) and after addition of 1.53 ml (7.16 mmol, 2.0 eq) of Boc₂O the mixture was stirred at room temperature for 19 h. The dioxane was removed under reduced pressure, the pH was checked (pH≧8), the mixture was washed twice with 10 ml each of MTBE, the aqueous phase was brought to pH=1 by addition of solid KHSO₄, extracted three times with 10 ml each of DCM, the combined organic phases were dried over magnesium sulfate and the solvent was removed at reduced pressure. 723 mg (3.19 mmol, 89%) of the Boc-protected acid 166 were obtained as a white solid (86% ee, d.r.: 18:1 syn:anti).

M (C₁₁H₁₉NO₄): 229.27 g mol⁻¹.

[α]²⁰_{D}: 36.6° (86% ee, d.r.: 18:1 syn:anti) (c=1.110, CHCl₃).

Melting point: 87.4° C.

¹H-NMR (300 MHz, CDCl₃): δ=1.07 (d, J 7.0, 3H, H7), 1.42 (s, 9H, H5), 2.77-2.61 (m, 1H, H6), 4.33 (dd, J 8.4, 4.5, 1H, H2), 5.01 (d, J 8.6, 1H, NH), 5.17-5.04 (m, 2H, H9), 5.74 (ddd, J 17.5, 10.3, 7.6, 1H, H8), 9.63 (s, 1H, COOH).

¹³C-NMR (75 MHz, CDCl₃): δ=15.16 (q, C7), 28.25 (q, C5), 40.31 (d, C6), 57.24 (d, C2), 80.21 (s, C4), 116.36 (t, C9), 138.44 (d, C8), 155.55 (s, C3), 176.42 (s, C1).

GC-MS: m/e=173 (3), 156 ([M]⁺-C₄H₉O, 2), 128 (7), 112 (17), 101 (3), 84 (7), 74(13), 57 (100). ([M]⁺-C₄H₃F₃NO₃, 100).

IR (FT-ATR): ^{˜}v=3313 (m, br), 3073 (m, br), 2974 (s), 2920 (s), 2546 (w, br), 1712 (s), 1503 (s), 1453 (s), 1390 (s), 1366 (s), 1250 (s), 1160 (s), 1090 (s), 1065 (s), 1013 (s), 995 (s), 918 (s), 856 (s), 776 (s), 700 (s), 663 (s).

HR-MS: (ESI, C₁₁H₁₉NNaO₄): 252.121±0.002 u (252.1212 u).

### Example 11

### But-2-yn-1-yl 2-(2,2,2-trifluoroacetamido)acetate (142)

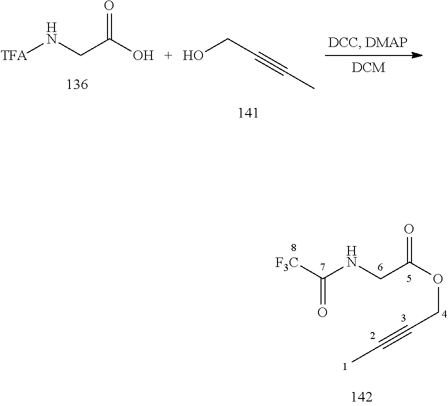

1.546 g (12.63 mmol, 0.3 eq.) of DMAP were added at 0° C. to a solution of 7.242 g (42.33 mmol, 1.0 eq.) of TFA glycine 136, 9.681 g (46.92 mmol, 1.1 eq.) of DCC and 3.5 ml (46.30 mmol, 1.1 eq.) of 2-butyn-1-ol (141) in 124 ml of DCM and the reaction mixture was stirred overnight at room temperature. The resulting urea was filtered off. The organic phase was washed with 120 ml each of water, 1 N HCl solution and saturated NaCl solution, dried over MgSO₄ and the solvent was removed at reduced pressure. The crude product was purified by means of column chromatography (EtOAc/CyHex 1:5) and 6.546 g (29.33 mmol, 69%) of the product 142 were obtained as a white/yellow solid.

M (C₈H₈F₃NO₃): 225.17 g mol⁻¹

R_{f}: 0.22 (1:5 EtOAc:CyHex).

Mp.: 68.1° C.

¹H-NMR (300 MHz, CDCl₃): δ [ppm]=1.82-1.84 (t, J=2.3 Hz, 3H, H1), 4.13-4.15 (d, J=5.2 Hz, 2H, H6), 4.72-4.74 (dd, J=2.1 Hz, 2.4 Hz, 2H, H4), 7.01 (s br, 1H, NH).

¹³C-NMR: (75 MHz, CDCl₃): δ [ppm]=3.70 (q, C1), 41.02 (d, C6), 54.31 (d, C4), 71.98 (s, C2), 84.44 (s, C3), 117.42 (s, C8), 157.06 (s, C7), 167.76 (s, C5).

GC-MS: m/e=223 ([M]⁺,1) 207 ([M]⁺-CH₃, 4), 154 (5), 126 (87), 97 (7), 78 (13), 69 (70), 53 (100).

IR: [cm⁻¹]: ^{˜}v=3296 (s), 3109 (w), 3000 (w), 2925 (w), 2856 (w), 2320 (w), 2245 (w), 1740 (s), 1701 (s), 1453 (w), 1420 (m), 1392 (m), 1352 (s), 1269 (m), 1200 (s), 1176 (s), 1153 (s), 1080 (w), 1017 (m), 952 (s), 850 (w), 799 (w), 729 (m), 696 (m).

### Example 12

### (Z)-But-2-en-1-yl 2-(2,2,2-trifluoroacetamido)acetate (143)

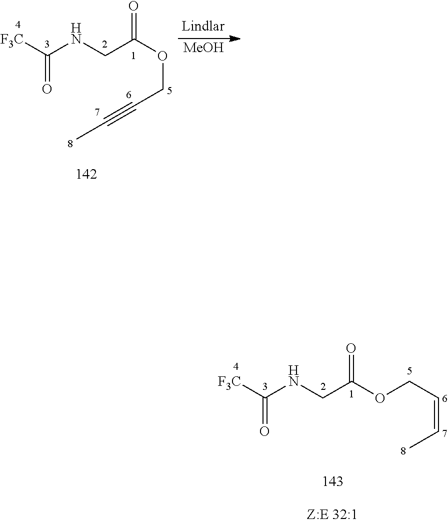

A suspension of 2.742 g (12.42 mmol, 1.0 eq.) of TFA glycine ester 142 and 203 mg (8%) of Lindlar cat in 30 ml of methanol was saturated with hydrogen. The reaction mixture was stirred at room temperature for 3 hours under H₂ (balloon) and the solvent was subsequently removed under reduced pressure. The residue was taken up in 15 ml of DCM and filtered through Celite. The solvent was again removed under reduced pressure. 2.496 g (11.08 mmol, 90%) of the product 143 were obtained as a slightly yellow solid.

M (C₈H₈F₃NO₃): 225.17 g mol⁻¹

R_{f}: 0.16 (EtOAc:CyHex 1:5).

Mp.: 34.6° C.

¹H-NMR (300 MHz, CDCl₃): δ [ppm]=1.68-1.70 (d, J=5.8 Hz, 3H, H8), 4.09-4.11 (d, J=4.9 Hz, 2H, H2), 4.72-4.74 (d, J=6.3 Hz, 2H, H5), 5.51-5.53 (d, J=6.6 Hz, 1H, H6), 5.73-5.79 (dd, J=10.7, 6.6 Hz, 1H, H7), 7.02 (s, 1H, NH).

¹³C-NMR: (75 MHz, CDCl₃): δ [ppm]=13.25 (q, C8), 41.48 (d, C2), 61.72 (d, C5), 113.81 (s, C4), 123.06 (t, C6), 131.20 (t, C7), 157.09 (s, C3), 168.32 (s, CI).

GC-MS: rm/e=225 ([M]⁺,1), 154 ([M]⁺-C₄H₇O, 2), 126 ([M]⁺-C₅H₇O₂, 49), 78 (11), 69 (29), 55 (([M]⁺-C₄H₃F₃NO₃, 100).

IR: [cm⁻¹]: ^{˜}v=3329 (m), 3106 (w), 3026 (w), 2946 (w), 1746 (m), 1714 (s), 1553 (m), 1412 (w), 1386 (w), 1373 (w), 1347 (w), 1280 (m), 1177 (s), 1073 (w), 1010 (m), 941 (m), 840 (w), 770 (w), 720 (w).

### Example 13

### (2S,3S)-3-Methyl 2-(2,2,2-trifluoroacetamido)acetate (144)

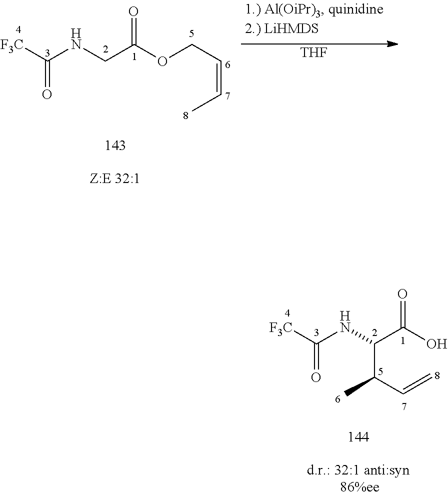

135 ml (135.00 mmol, 5.0 eq.) of LiHMDS solution in THF Cl M, Aldrich) were added dropwise over 110 min at −78° C. to a solution of 6.070 g (26.96 mmol, 1.0 eq.) of the ester 143, 6.067 g (29.70 mmol, 1.1 eq.) of Al(OiPr)₃ and 19.165 g (59.44 mmol, 2.2 eq.) of quinidine in 270 ml of THF and the solution was slowly warmed to room temperature with stirring overnight. After addition of 1000 ml of half-saturated NaHCO₃ solution, the mixture was washed two times with 1000 ml each of MTBE, the aqueous phase in the separating funnel was brought to pH=1 by addition of solid KHSO₄ and it was subsequently extracted three times with 1000 ml each of DCM. The combined DCM phases were dried over MgSO₄ and the solvent was removed at reduced pressure. 963 mg (4.28 mmol, 16%) of the rearranged product 144 were obtained as a brown-orange oil (86% ee).

M (C₈H₈F₃NO₃): 225.17 g mol⁻¹

[α]²⁰_{D}: +31.4° (c=0.555, CHCl₃).

¹H-NMR (300 MHz, CDCl₃): δ [ppm]=1.06-1.08 (d, J=6.6 Hz, 3H, H6), 2.76-2.96 (m, 1H, H5), 4.45-4.71 (m, 1H, H2), 5.12 (d, J=11.6 Hz, 2H, H8), 5.65-5.85 (m, 1H, H7), 6.98 (s, 1H, NH), 10.76 (s, 1H, COOH).

¹³C-NMR: (75 MHz, CDCl₃): δ [ppm]=14.91 (q, C6), 40.06 (d, C5), 56.14 (d, C2), 109.84/113.65/117.24/121.28 (s, C4, J=285.75 Hz, 269.25 Hz, 302.25 Hz), 117.53 (t, C8), 136.95 (d, C7), 156.28/156.78/157.28/157.78 (s, C3, J=37.5 Hz, 37.5 Hz, 37.5 Hz), 179.93 (s, C1).

IR: [cm⁻¹]=3284 (m), 3087 (m), 2976 (m), 2624 (w), 1711 (s), 1650 (m), 1643 (m), 1555 (s), 1536 (s), 1454 (m), 1416 (m), 1684 (m), 1356 (m), 1278 (s), 1206 (s), 1162 (s), 1023 (m), 994 (s), 924 (s), 860 (m), 803 (m), 770 (s), 727 (s), 696 (s).

### Example 14

### (2S,3S)-2-((tert-Butoxycarbonyl)amino)-3-methylpent-4-enoic acid (140)

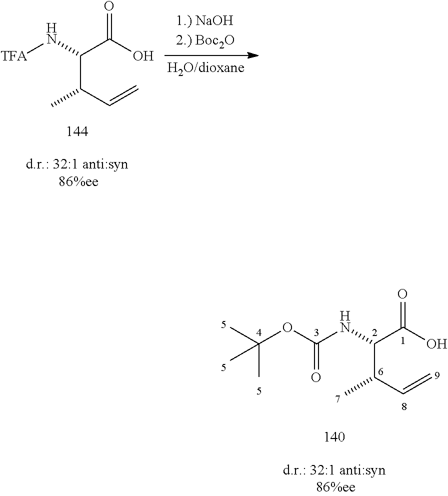

10.6 ml (10.53 mmol, 2.5 eq.) of a 1 N aqueous NaOH solution were added to a solution of 947 mg (4.21 mmol, 1.0 eq.) of the TFA-protected acid 144 in 10.6 ml of dioxane and the solution was stirred at room temperature for 18 h. The pH was checked (pH≧8) and after addition of 1.82 ml (8.42 mmol, 2.0 eq.) of Boc₂O the mixture was stirred at room temperature for 5 h. The dioxane was removed under reduced pressure and the pH (pH≧8) was checked, the mixture was washed twice with 30 ml each of MTBE, the aqueous phase was brought to pH=1 by addition of solid KHSO₄, extracted three times with 80 ml each of DCM and dried over MgSO₄. The solvent was removed under reduced pressure and 943 mg (98%, 4.11 mmol) of the Boc-protected acid 140 were obtained as a brown-orange oil.

M (C₁₁H₁₉F₃NO₄): 229.27 g mol⁻¹

[α]²⁰_{D}: +9.9° (c=0.555, CHCl₃).

IR: [cm⁻¹]: =3306 (w), 3080 (m), 2976 (s), 2926 (m), 2553 (w), 1786 (m), 1714 (s), 1513 (s), 1446 (s), 1416 (s), 1367 (s), 1286 (s), 1253 (s), 1151 (s), 1053 (s), 1015 (s), 990 (s), 920 (s), 855 (s), 778 (s), 730 (s).

¹H-NMR (300 MHz, CDCl₃): δ [ppm]=1.13 (d, J=6.9 Hz, 3H, H7), 1.45 (s, 9H, H5), 2.77 (m, 1H, H6), 4.31 (dd, J=8.5 Hz, 4.6 Hz, 1H, H2), 5.03 (d, J=8.7 Hz, 1H, NH), 5.12 (s, 1H, H4′), 5.16 (d, J=3.9 Hz, 1H), 5.72 (ddd, J=17.4 Hz, 10.0 Hz, 7.3 Hz, 1H), 9.75 (s, 1H, COOH).

¹³C-NMR: (75 MHz, CDCl₃): δ [ppm]=18.46 (q, C7), 27.93 (q, C5), 39.98 (t, C6), 57.76 (t, C2), 80.51 (q, C4), 117.23 (d, C9), 137.52 (t, C8), 156.15 (s, C3), 176.37 (s, C1).

### Example 15

### (2S,5R)-tert-Butyl 1-((2S,3R)-2-((tert-butoxycarbonyl)amino)-3-methylpent-4-enyl)-5-vinylpyrrolidine-2-carboxylate (115)

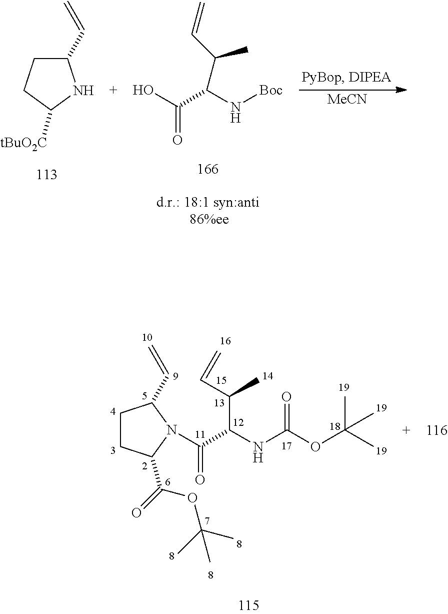

A solution of 1.311 g (2.52 mmol, 1.1 eq) of PyBOP in 11 ml of MeCN was added dropwise at 0° C. to a solution of 525 mg (2.29 mmol, 1.0 eq) of the acid 166, 452 mg (2.29 mmol, 1.0 eq) of the amine 113 and 1.14 ml (6.87 mmol, 3.0 eq) of diisopropylethylamine in 15 ml of MeCN. The solution was stirred overnight at room temperature and the solvent was removed at reduced pressure. The residue was taken up with 50 ml of MTBE and 15 ml of water, the aqueous phase was extracted two more times with 30 ml each of MTBE and the combined organic phases were dried over magnesium sulfate. The solvent was removed at reduced pressure and by column-chromatographic purification on silica gel with EtOAc/CyHex 1:3 698 mg (1.71 mmol, 75%) of the product 115, which contained traces of a diastereomer 116, were obtained as a slightly yellowish oil.

M (C₂₂H₃₆N₂O₅): 408.53 g mol⁻¹.

TLC: R_{f}=0.37 (EtOAc/CyHex 1:3).

[α]²⁰_{D}: −51.2° (c=2.400, CHCl₃).

¹H-NMR (600 MHz, CDCl₃): δ=1.37, 1.42 (2×s, 9H, H8, H19), 1.82-1.76 (m, 1H, H4′), 1.97-1.88 (m, 1H, H3′), 2.17-2.05 (m, 2H, H3, H4), 2.62-2.53 (m, 1H, H13), 4.32 (t, J 7.8, 1H, H2), 4.40 (dd, J 9.1, 6.3, 1H, H12), 4.70 (t, J 6.9, 1H, H5), 5.01-4.96 (m, 2H, H16), 5.04 (d, J 9.5, 1H, NH), 5.15 (d, J 10.3, 1H, H10′), 5.45 (d, J 17.2, 1H, H10), 5.76 (ddd, J 17.0, 9.6, 7.7, 1H, H15), 5.99-5.92 (m, 1H, H9).

¹³C-NMR (150 MHz, CDCl₃): δ=13.95 (q, C14), 27.18 (t, C3), 27.86, 28.24 (q, C8, 19), 32.71 (t, C4), 39.78 (d, C13), 54.33 (d, C12), 61.00 (d, C2), 61.22 (d, C5), 79.48, 81.13 (2×q, C7, C18), 115.15 (t, C16), 116.62 (t, C10), 138.82 (d, C9), 139.57 (d, C15), 155.55 (s, C17), 171.08 (s, C6), 171.42 (s, C11).

IR (FT-ATR): ^{˜}v=3437 (w), 3298 (w), 3078 (w) 2974 (m), 2930 (m), 2874 (w), 1706 (s), 1636 (s) 1496 (s), 1425 (s), 1390 (s), 1364 (s), 1287 (s), 1250 (s) 1209 (s), 1151 (s), 1089 (m), 1068 (m), 1041 (m), 987 (s), 913 (s), 874 (m), 843 (m), 769 (m), 735 (m), 666 (m).

### Example 16

### (3S,6S,7R,9aR)-tert-Butyl 6-((tert-butoxycarbonyl)amino)-7-methyl-5-oxo-2,3,5,6,7,9a-hexahydro-1H-pyrrolo[1,2-a]azepine-3-carboxylate (117)

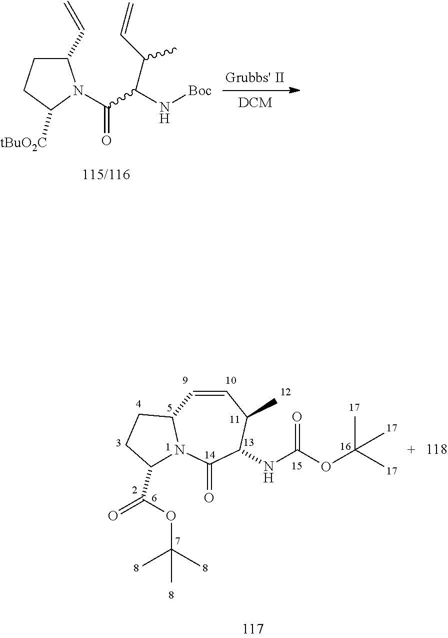

A solution of 312 mg (0.76 mmol, 1.0 eq) of the dipeptide mixture 115/116 and 65 mg (0.08 mmol, 0.1 eq) of Grubbs II in 15 ml of DCM was heated to reflux under argon for 20 h at 50° C. 0.4 ml of DMSO was then added and the solvent was removed to 80 mbar at reduced pressure. By chromatography on silica gel with EtOAc/CyHex 1:3 as eluent 241 mg (0.63 mmol, 83%) of the product 117 were obtained as a gray solid. 3 mg (0.01 mmol, 1%) 118 could furthermore be isolated.

M (C₂₀H₃₂N₂O₅): 380.48 g mol⁻¹.

TLC: R_{f}=0.61 (EtOAc/CyHex 1:1); 0.33 (EtOAc/CyHex 1:3).

[α]²⁰_{D}: −145.7° (c=0.675, CHCl₃).

Melting point: 143.6° C.

¹H-NMR (600 MHz, CDCl₃): δ=1.11 (d, J 7.2, 3H, H12), 1.38, 1.39 (2×s, 9H, H8, H17), 1.86-1.77 (m, 1H, H4′), 2.04-1.96 (m, 2H, H3), 2.30-2.23 (m, 1H, H4), 2.41-2.33 (m, 1H, H11), 4.45 (dd, J 7.3, 3.5, 1H, H2), 4.50 (dd, J 10.8, 7.1, 1H, H13), 4.58 (t, J 6.7, 1H, H5), 5.49 (d, J 11.7, 1H, H9), 5.56 (dt, J 11.6, 2.3, 1H, H10).

¹³C-NMR (150 MHz, CDCl₃): δ=18.78 (q, C12), 27.62 (t, C3), 27.84, 28.27 (2×q, C8, C17), 32.90 (t, C4), 36.49 (d, C11), 54.99 (d, C13), 55.59 (d, C5), 60.81 (d, C2), 79.27, 81.34 (2×s, C7, C16), 127.57 (d, C9), 134.62 (d, C10), 156.02 (s, C15), 170.20 (s, C14), 170.56 (s, C6).

IR (FT-ATR): ^{˜}v=3399 (w), 2979 (m), 2930 (w), 2873 (w), 1715 (s), 1649 (s), 1493 (s), 1438 (s), 1391 (m), 1365 (s), 1316 (w), 1291 (w), 1251 (m), 1223 (w), 1153 (s), 1071 (w), 1044 (w), 1023 (w), 980 (w), 947 (w), 910 (w), 879 (w), 848 (w), 806 (w), 760 (w), 684 (w), 660 (w).

### Example 17

### (3S,6S,7R,9aR)-6-((((9H-Fluoren-9-yl)methoxy)carbonyl)amino)-7-methyl-5-oxo-2,3,5,6,7,9a-hexahydro-1H-pyrrolo[1,2-a]azepine-3-carboxylic acid (85)

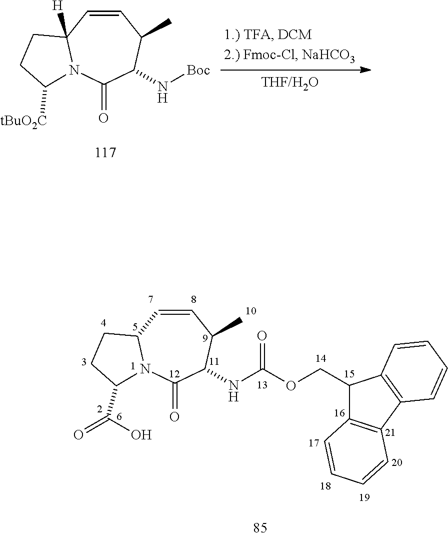

2 ml of TFA were added at 0° C. to a solution of 125 mg (0.33 mmol, 1.0 eq) of the dipeptide 117 in 2 ml of DCM and the mixture was subsequently stirred at room temperature for 1 h. The solvent was removed at reduced pressure, the residue was taken up in 3 ml of half-saturated NaHCO₃ soln. and the pH was checked (pH≧8). A solution of 128 mg (0.49 mmol, 1.5 eq) of Fmoc-Cl in 1.5 ml of THF was then added at 0° C. and the mixture was stirred overnight at room temperature. After addition of 5 ml of DCM, the solution was brought to pH=1 at 0° C. with 1 N HCl soln., the phases were separated and the aqueous phase was extracted twice with DCM. The combined organic phases were dried over magnesium sulfate, the solvent was removed at reduced pressure and 77 mg (0.17 mmol, 52%) of the Fmoc-protected product 85 were obtained by chromatography on silica gel with DCM/MeOH 15:1 and 1 vol % AcOH as eluent.

M (C₂₆H₂₆N₂O₅): 446.50 g mol⁻¹.

TLC: R_{f}=0.20 (DCM/MeOH 15:1).

[α]²⁰_{D}: −152.7° (c=0.345, CHCl₃).

Melting point: 105.3° C.

¹H-NMR (500 MHz, CDCl₃): δ=1.12 (d, J 7.1, 3H, H10), 1.95-1.79 (m, 1H, H4′), 2.08-1.97 (m, 1H, H3′), 2.25-2.16 (m, 1H, H3), 2.35-2.26 (m, 1H, H4), 2.47 (s br, 1H, H9), 4.21 (t, J 7.3, 1H, H15), 4.39-4.28 (m, 2H, H14), 4.74-4.57 (m, 3H, H2, H5, H11), 5.49 (d, J 11.7, 1H, H8), 5.55 (d, J 11.7, 1H, H7), 6.21 (d, J 8.9, 1H, NH), 7.32-7.26 (m, 2H, H18), 7.41-7.35 (m, 2H, H19), 7.64-7.57 (m, 2H, H17), 7.74 (d, J 7.5, 2H, H₂O), 10.53 (s, 1H, COOH).

¹³C-NMR (125 MHz, CDCl₃): δ=18.75 (q, C10), 26.89 (t, C3), 32.92 (t, C4), 35.78 (d, C9), 47.02 (d, C15), 55.60 (d, C11), 55.91 (d, C5), 60.32 (d, C2), 67.10 (t, C14), 119.83 (d, C20), 125.12 (d, C17), 126.96 (2×d, C17, C8), 127.57 (d, C19), 134.55 (d, C7), 141.14 (s, C21), 143.61/143.83 (s, C16), 156.61 (s, C13), 171.57 (s, C12), 174.42 (s, C6).

IR (FT-ATR): ^{˜}v=3284 (w), 3015 (w), 2971 (w), 2873 (w), 1715 (s), 1642 (s), 1530 (m), 1447 (s), 1319 (m), 1250 (s), 1220 (m), 1186 (m), 1104 (w), 1083 (m), 1033 (m), 979 (w), 943 (w), 871 (w), 830 (w), 757 (s), 740 (s), 666 (w).

HR-MS: ((−)ESI, C₂₆H₂₅N₂O₅): 445.17581±0.00001 u (445.17580 u).

### Example 18

### (2S,5R)-tert-Butyl 1-((2S,3S)-2-((tert-butoxycarbonyl)amino)-3-methylpent-4-enoyl)-5-vinylpyrrolidine-2-carboxylate (epi-115)

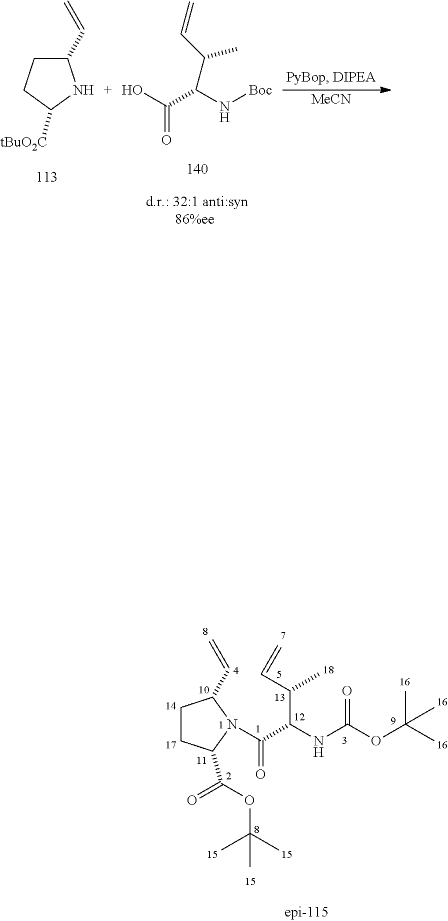

A solution of 1.253 g (2.41 mmol, 1.1 eq.) of PyBOP in 11 ml of MeCN was added dropwise at 0° C. to a solution of 499 mg (2.17 mmol, 1.0 eq.) of the acid 140, 430 mg (2.17 mmol, 1.0 eq.) of the amine 113 and 1.08 ml (6.53 mmol, 3.0 eq.) of diisopropylethylamine in 14 ml of MeCN. The solution was stirred overnight at room temperature and the solvent was removed at reduced pressure. The residue was taken up in 50 ml of MTBE and 15 ml of water, the aqueous phase was extracted twice with 30 ml each of MTBE and the combined organic phases were dried over MgSO₄. The solvent was removed under reduced pressure and by column-chromatographic purification on silica gel with EtOAc:CyHex 1:3 526 mg (1.29 mmol, 66%) of the product epi-115 were obtained as a yellowish oil.

M (C₂₂H₃₆N₂O₅): 408.53 g mol⁻¹.

R_{f}: 0.32 (1:3 EtOAc:CyHex).

[α]²³_{D}: −55.7° (c=0.555, CHCl₃).

¹H-NMR (500 MHz, CDCl₃): δ [ppm]=0.92-1.05 (m, 3H, H18), 1.37 (t, J=9.7 Hz, 18H, H15/16), 1.74-1.84 (m, 1H, H14), 1.86-1.98 (m, 1H, H17), 2.07-2.17 (m, 1H, H14/17), 2.47-2.61 (m, 1H, H13), 4.26 (m, 1H, H12), 4.34 (t, J=7.9 Hz, 1H, H11), 4.76 (t, J=6.9 Hz, 1H, H10), 4.86 (d, J=9.7 Hz, 1H, NH), 4.98-5.06 (m, 2H, H7), 5.17 (d, J=10.4 Hz, 1H, H6), 5.49 (d, J=17.3 Hz, 1H, H6′), 5.59-5.80 (m, 1H, H5), 5.99 (ddd, J=17.6 Hz, 10.3 Hz, 7.6 Hz, 1H, H4).

¹³C-NMR: (125 MHz, CDCl₃): δ [ppm]=16.73 (q, C18), 27.33 (d, C17), 27.96 (s, C15/16), 28.31 (s, C15/16), 32.65 (d, C14), 40.59 (t, C13), 54.86 (t, C12), 60.97 (t, C11), 61.26 (t, C10), 79.52 (s, C8/9), 81.26 (s, C8/9), 116.16 (d, C7), 116.55 (d, C6), 138.95 (t, C5), 139.16 (t, C4), 155.61 (s, C3), 171.17 (s, C2), 171.64 (s, C1).

IR: [cm⁻¹]: =3433 (w), 3300 (m), 3073 (w), 2973 (s), 2920 (s), 2866 (m), 1736 (s), 1706 (s), 1634 (s), 1510 (s), 1423 (s), 1383 (s), 1363 (m), 1290 (s), 1250 (w), 1164 (s), 1083 (s), 1066 (s), 993 (s), 916 (s), 873 (s), 840 (s), 776 (s), 730 (s).

### Example 19

### (3S,6S,7S,9aR)-tert-Butyl 6-((tert-Butoxycarbonyl)amino)-7-methyl-5-oxo-2,3,5,6,7,9a-hexahydro-1H-pyrrolo[1,2-a]azepine-3-carboxylate (epi-117)

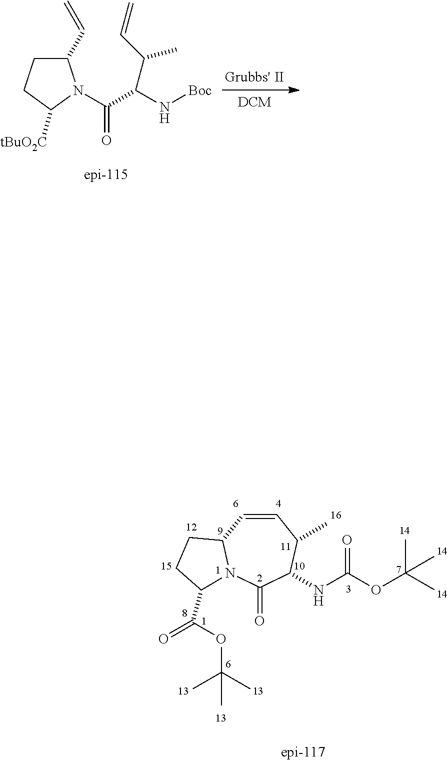

A solution of 300 mg (0.734 mmol, 1.0 eq.) of the dipeptide 115 and 62 mg (0.0734 mmol, 0.1 eq.) of Grubbs II in 14.5 ml of DCM was heated to reflux at 50° C. under argon for 20 h. 0.4 ml of DMSO was then added and the solvent was removed at reduced pressure. By chromatography on silica gel with EtOAc/CyHex 1:2 as eluent, 249 mg (0.654 mmol, 89%) of the product 117 were obtained as a brown oil.

M (C₂₀H₃₂N₂O₅): 380.48 g mol⁻¹.

R_{f}: 0.36 (1:2 EtOAc:CyHex).

[α]²³_{D}: −83.0° (c=0.495, CHCl₃).

¹H-NMR (600 MHz, CDCl₃): δ [ppm]=0.99-1.00 (d, J=7.1 Hz, 1H, H16), 1.39 (s, 18H, H13/14), 1.75-1.85 (m, 2H, H12), 2.17-2.34 (m, 2H, H15), 2.56-2.69 (m, 1H, H11), 4.47-4.50 (m, 1H, H9), 4.51-4.54 (m, 1H, H8), 4.74-4.80 (m, 1H, H10), 5.46 (d, J=11.6 Hz, 1H, H5), 5.69-5.78 (m, 1H, H4).

¹³C-NMR: (150 MHz, CDCl₃): δ [ppm]=14.17 (q, C16), 27.53 (d, C15), 27.81 (q, C13/14), 28.20 (q, C13/14), 33.02 (d, C12), 35.83 (t, C11), 54.20 (t, C10), 56.40 (t, C9), 60.59 (t, C8), 79.25, 81.26 (s, C6/C7), 126.59 (t, C5), 133.75 (t, C4), 155.00 (s, C3), 168.77 (s, C2), 170.59 (s, C1).

IR: [cm⁻¹]: =3400 (m), 3340 (w), 2974 (s), 2933 (m), 2880 (w), 1711 (s), 1660 (s), 1490 (s), 1426 (s), 1365 (s), 1246 (s), 1223 (s), 1153 (s), 1053 (s), 1020 (m), 960 (m), 880 (m), 843 (m), 816 (m), 754 (s), 660 (m).

### Example 20

### (3S,6S,7S,9aR)-6-((((9H-fluoren-9-yl)methoxy)carbonyl)amino)-7-methyl-5-oxo-2,3,5,6,7,9a-hexahydro-1H-pyrrolo[1,2-a]azepine-3-carboxylic acid (86)

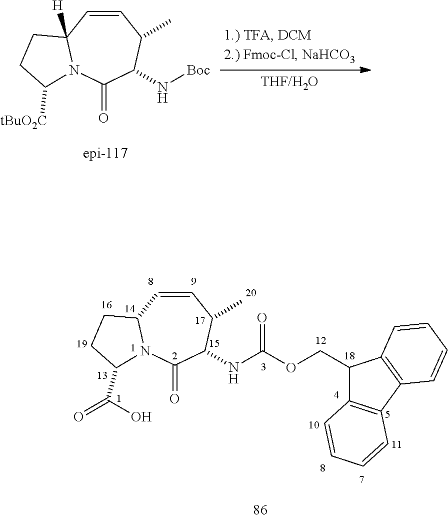

2 ml of TFA were added at 0° C. to a solution of 249 mg (0.654 mmol, 1.0 eq.) of the dipeptide 117 in 2 ml of DCM and the mixture was subsequently stirred at room temperature for 1 h. The solvent was removed at reduced pressure, the residue was taken up in 3 ml of half-saturated NaHCO₃ solution (pH≧8). A solution of 254 mg (0.982 mmol, 1.5 eq.) of Fmoc-Cl in 3 ml of THF was then added at 0° C. and the mixture was stirred overnight at room temperature. After addition of 5 ml of DCM, the solution was brought to pH=1 at 0° C. with 1 N HCl solution, the phases were separated and the aqueous phase was extracted 2× with DCM. The combined organic phases were dried over MgSO₄, the solvent was removed under reduced pressure and the residue was purified by chromatography on a Grace Reveleris™ system using DCM/MeOH. 189 mg (0.423 mmol, 65%) of the product 86 were obtained as a pale brown solid.

M (C₂₆H₂₆N₂O₅): 446.50 g mol⁻¹.

R_{f}: 0.25 (15:1 DCM:MeOH).

[α]²³_{D}: −61.3° (c=0.240, CHCl₃).

Mp.: 136.7° C.

¹H-NMR (600 MHz, CDCl₃): δ [ppm]=0.99-1.00 (d, J=7.1 Hz, 2H, H₂O), 1.23-1.25 (t, J=7.1 Hz, 1H, H20′), 1.84-1.93 (m, 1H, H18′), 1.96-2.02 (m, 1H, H19′), 2.30-2.33 (m, 1H, H18/H19), 2.70 (s, 1H, H17), 4.19-4.21 (t, J=5.6 Hz, 1H, H16), 4.35-4.36 (d, J=7.2 Hz, 1H, H12), 4.57 (s, 1H, H14), 4.71-4.73 (d, J=8.3 Hz, 1H, H13), 4.91-4.92 (m, 1H, H15), 5.46-5.48 (d, J=11.6 Hz, 1H, H9), 5.72-5.75 (m, 1H, H6), 6.15-6.16 (d, J=6.7 Hz, 1H, NH), 7.28-7.31 (t, J=7.2 Hz, 1H, H8), 7.37-7.39 (t, J=7.2 Hz, 1H, H7), 7.58-7.59 (d, J=7.5 Hz, 1H, H10), 7.74-7.75 (d, J=7.4 Hz, 1H, H11).

¹³C-NMR: (150 MHz, CDCl₃): δ [ppm]=14.35 (q, C20), 26.63 (d, C19), 33.54 (d, C18), 35.90 (t, C17), 47.25 (t, C16), 54.88 (t, C15), 57.27 (t, C14), 60.61 (t, C13), 67.23 (d, C12), 120.12 (t, C11), 125.26 (t, C10), 126.27 (t, C9), 127.21 (t, C8), 127.86 (t, C7), 134.12 (t, C6), 141.43 (s, C5), 143.95 (s, C4), 155.62 (s, C3), 171.10 (s, C2), 173.51 (s, C1).

IR: [cm⁻¹]: =3300 (m), 3066 (w), 2974 (s), 2926 (m), 1704 (s), 1640 (s), 1506 (s), 1443 (s), 1390 (s), 1365 (s), 1303 (s), 1243 (s), 1154 (s), 1073 (m), 1040 (m), 993 (m), 916 (m), 856 (m), 758 (s), 740 (s), 693 (m).

### Abbreviations

abs. absolute

eq. equivalent(s)

Ar aryl

ATR attenuated total internal reflectance

9-BBN 9-borabicyclononane

calc. calculated

Bn benzyl

Boc tert-butoxycarbonyl

Boc₂O di-tert-butyl dicarbonate (Boc anhydride)

CH cyclohexane

Cy cyclohexyl

TLC thin-layer chromatogram

DCE dichloroethane (1,2-)

DCM dichloromethane

DIBAL-H diisobutylaluminum hydride

DIC diisopropylcarbodiimide

DIP direct inlet (mass spectrometry)

DIPEA diisopropylethylamine

DMAP 4-N,N-dimethylaminopyridine

DMF N,N-dimethylformamide

DMP 2,2-dimethoxypropane

DMS dimethyl sulfide

DMSO dimethyl sulfoxide

dr diastereomeric ratio

EA ethyl acetate

ee enantiomeric excess

EI electron impact ionization

ESI electron spray ionization

Et ethyl

Et₂O diethyl ether

EtOH ethanol

EVH1 ena-VASP-homology-1 domain

FGI functional group chemistry (function group inversion)

Fmoc fluorenyl-9-methoxycarbonyl

FMP Forschungsinstitut für Molekulare Pharmakologie [Research

Institute for Molecular Pharmacology] (Berlin-Buch)

GC-MS gas chromatography-mass spectroscopy

fnd. found

satd. saturated

HPLC high performance liquid chromatography

HRMS high-resolution mass spectrometry

IR infrared spectroscopy

conc. concentrated

LiHMDS lithium hexamethyldisilazide

M molar mass

MCPBA meta-chloroperbenzoic acid

Me methyl

MeOH methanol

Ms mesyl (methanesulfonyl)

MS mass spectrometry

MTBE methyl tert-butyl ether

NaHMDS sodium hexamethyldisilazide

NME N-methylephedrine

NMR nuclear magnetic resonance spectroscopy

NOE nuclear overhauser effect

o ortho

Ph phenyl

PHI polyproline helix type II

POM-Cl chloromethyl pivaloate

PPTS pyridinium para-toluenesulfonate

RCM ring closure metathesis

R_{f} retention factor

[Ru]_{II} Grubbs-II catalyst 81

[Ru]_{gr} modified (green) Grubbs-Hoveyda catalyst after Blechert 82

s secondary

Mp. melting point

Su succinimide

TBAF tetrabutylammonium fluoride

TBS tert-butyldimethylsilyl

t-Bu tert-butyl

Tf trifluoromethanesulfonyl

TFA trifluoroacetic acid

THF tetrahydrofuran

TMEDA tetramethylethylenediamine

TMS trimethylsilyl

TMS OTf trimethylsilyl trifluoromethanesulfonate

TPS tert-butyldiphenylsilyl

Ts tosyl (para-toluenesulfonyl)

TsOH para-toluenesulfonic acid

Z benzyloxycarbonyl- (also Cbz)

A letter code and three-letter code of natural proteinogenic amino acids:

| | | |
|---|---|---|
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

The present invention is not limited to embodiments described herein; reference should be had to the appended claims.

What is claimed is:

1. A compound comprising a general formula 1 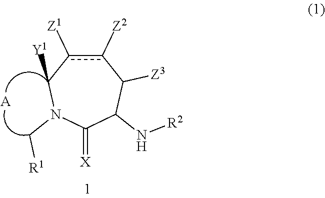
comprising a saturated or an unsaturated central seven-membered ring,
wherein,
X is at least one of O and S;
A is a ring bridge;
Y¹ is at least one of H, alkyl, fluoroalkyl, aryl and heteroaryl;
Z¹, Z², Z³ are, individually or alternatively, at least one of H, carbonyl, OH, O-alkyl, O-acyl, N—R¹R² (where R¹ or R² are, individually or alternatively, at least one of H, alkyl, acyl and sulfonyl), alkyl, acyl, fluoroalkyl, aryl, and heteroaryl;
R¹ is at least one of alkyl, acyl, alkoxycarbonyl, aryloxycarbonyl and aminocarbonyl; and
R² is at least one of H, alkyl, aryl, alkylcarbonyl, arylcarbonyl, alkoxycarbonyl, aminocarbonyl, aryloxycarbonyl, alkylsulfonyl, arylsulfonyl, aminoacyl and peptidyl.

2. The compound as recited in claim 1, wherein A comprises a 5 or a 6 atom cyclic ring, with members of the 5 or the 6 atom cyclic ring being selected from at least one of C, O, S and N atoms.

3. The compound as recited in claim 2 comprising a general formula 2 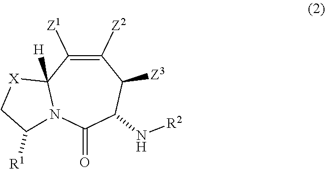
wherein,
Z¹, Z², Z³ are, individually or alternatively, at least one of H, carbonyl, OH, O-alkyl, O-acyl, N—R¹R² (where R¹ or R² are, individually or alternatively, at least one of H, alkyl, acyl and sulfonyl), alkyl, acyl, fluoroalkyl, aryl, and heteroaryl;
R¹, R² are at least one of alkyl, acyl, hetaryl, and sulfonyl; and
X is at least one of —CH₂—, —O—, —S— and —NH—R.

4. The compound as recited in claim 3 comprising a general formula 3 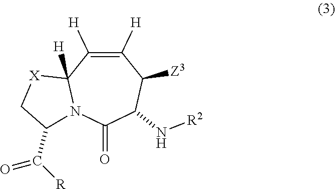
wherein,
X is at least one of —CH₂—, —O—, and —S—;
Z³ is at least one of H, carbonyl, OH, O-alkyl, O-acyl, N—R¹R² (where R¹ or R² are, individually or alternatively, at least one of H, alkyl, acyl and sulfonyl), alkyl, acyl, fluoroalkyl, aryl, and heteroaryl, and Z³ has a configuration of the general formula 2;
R is at least one of NH—R″, and —O—R″, with R″ being at least one of peptidyl, substituted alkyls, and hetaryl; and
R² is at least one of acyl, peptidyl, and sulfonyl.

5. A method of using the compound as recited in claim 1 as a pharmaceutically active compound, the method comprising:
providing the compound as recited in claim 1; and
using the compound recited in claim 1 as a pharmaceutically active compound.

6. A method of using the compound as recited in claim 1 as a ligand for a domain, the method comprising:
providing the compound as recited in claim 1; and
using the compound as a ligand for a domain;
wherein, the domain is selected from the group comprising Src-homology-3 domains, WW domains, Ena-VASP-homology-1 domains, GYF domains, UEV domains, and profilin.

7. A method of using the compound as recited in claim 1 as a polyproline mimetic, the method comprising:
providing the compound as recited in claim 1; and
using the compound as a polyproline mimetic.

8. A pharmaceutical composition comprising the compound as recited in claim 1 and a pharmaceutically tolerable carrier.

9. The pharmaceutical composition as recited in claim 8, wherein the pharmaceutically tolerable carrier is at least one of a filler, an extender, a binder, a humectant, a solution retardant, a disintegrant, an absorption accelerator, a wetting agent, an absorbent and a glidant.

10. A method of using the pharmaceutical composition as recited in claim 8 for the treatment of diseases that are associated with a modification of intracellular signal transduction processes mediated by poly-proline helix structures, the method comprising:
providing the pharmaceutical composition as recited in claim 8; and
using the pharmaceutical composition to treat diseases associated with a modification of intracellular signal transduction processes mediated by poly-proline helix structures.

11. The method as recited in claim 10, wherein the diseases associated with a modification of intracellular signal transduction processes mediated by poly-proline helix structures include a bacterial infectious disease, a neurodegenerative disease and a tumor.

12. The method as recited in claim 11, wherein the bacterial infectious disease is caused by a bacteria selected from at least one of Legionella, streptococci, staphylococci, Klebsiella, Hemophilis influenzae, Rickettsia (petechiae), mycobacteria, mycoplasmas, ureaplasmas, Neisseria (meningitis, Waterhouse-Friedrichsen syndrome, gonorrhoea), pseudomonads, Bordetella (pertussis), Corynebacteria (diphtheria), Chlamydia, Campylobacter (diarrhoea), Escherichia coli, Proteus, Salmonella, Shigella, Yersinia, vibrios, Enterococci, Clostridia, Borrelia, Treponema pallidum, Brucellae, Francisellae, Leptospira, and Listeria.

13. The method of using as recited in claim 12, wherein the Listeria are selected from at least one of L. monocytogenes Sv1/2a, L. monocytogenes Sv4b F2365, L. monocytogenes Sv4b H7858, 178 contigs, L. monocytogenes Sv1/2a F6854, 133 contigs, L. monocytogenes Sv4b, L. monocytogenes Sv4a, L. innocua Sv6a, L. welshimeri Sv6b, L. seeligeri Sv1/2b, and L. ivanovii Sv5.

14. The method of using as recited in claim 11, wherein the neurodegenerative disease is selected from at least one of Alzheimer's disease, Parkinson's disease, Huntington's disease, and amyotrophic lateral sclerosis (ALS).

15. The method of using as recited in claim 11, wherein the tumor is selected from at least one of a carcinoma, a sarcoma, a neuroendocrine tumor, a hemooncological tumor, a dysontogenetic tumor, and a mixed tumor.

16. The method of using as recited in claim 11, wherein the tumor is selected from:
a tumor of the ear, nose and throat region comprising tumors of the inner nose, of the paranasal sinuses, of the nasopharynx, of the lips, of the oral cavity, of the oropharynx, of the larynx, of the hypopharynx, of the ear, of the salivary glands, and paraganglioma,
tumors of the lung comprising non-small cell bronchial carcinomas, small cell bronchial carcinomas, tumors of the mediastinum,
tumors of the gastrointestinal tract comprising tumors of the esophagus, of the stomach, of the pancreas, of the liver, of the gallbladder and of the bile ducts, of the small intestine, colon and rectal carcinomas and anal carcinomas,
urogenital tumors comprising tumors of the kidneys, of the ureter, of the bladder, of the prostate, of the urethra, of the penis and of the gonads,
gynecological tumors comprising tumors of the cervix, of the vagina, of the vulva, uterine carcinoma, malignant trophoblastic disease, ovarian carcinoma, tumors of the oviduct (Fallopian tube), tumors of the abdominal cavity, mammary carcinomas,
tumors of endocrine organs comprising tumors of the thyroid, of the parathyroid, of the adrenal cortex, endocrine pancreatic tumors, carcinoid tumors and carcinoid syndrome,
multiple endocrine neoplasias, bone and soft tissue sarcomas, mesotheliomas, skin tumors, melanomas comprising cutaneous and intraocular melanomas,
tumors of the central nervous system,
tumors in childhood comprising retinoblastoma, Wilms' tumor, neurofibromatosis, neuroblastoma, Ewing's sarcoma tumor family, rhabdomyosarcoma, lymphomas comprising non-Hodgkin lymphoma, cutaneous T-cell lymphomas, primary lymphomas of the central nervous system, Hodgkin's disease,
leukemias comprising acute leukemias, chronic myeloid and lymphatic leukemias,
plasma cell neoplasms,
myelodysplastic syndromes, paraneoplastic syndromes, metastases without known primary tumor (CUP syndrome), peritoneal carcinomatosis,
immunosuppression-related malignancy comprising AIDS-related malignancies, Kaposi's sarcoma, AIDS-associated lymphomas, AIDS-associated lymphomas of the central nervous system, AIDS-associated Hodgkin's disease and AIDS-associated anogenital tumors,
transplantation-related malignancies, and
metastasized tumors comprising cerebral metastases, pulmonary metastases, hepatic metastases, bone metastases, pleural and pericardial metastases and malignant ascites.

17. The method of using as recited in claim 8, wherein the pharmaceutical composition is provided as at least one of a gel, a powder, a tablet, a delayed release tablet, a premix, an emulsion, an infusion formulation, a drop, a concentrate, a granule, a syrup, a pellet, a boli, a capsule, an aerosol, a spray, and an inhalate.

18. The method of using as recited in claim 8, further comprising:
providing the pharmaceutical composition in a preparation,
wherein a concentration of the pharmaceutical composition in the preparation is from 0.1 to 99.5 wt.-%.

19. The method of using as recited in claim 18, further comprising:
using the preparation at least one of orally, subcutaneously, intravenously, intramuscularly, intraperitoneally, and topically.

20. The method of using as recited in claim 8,
using the pharmaceutical composition in an amount of 0.05 to 500 mg/kg of a body weight every 24 hours.

21. The method of using as recited in claim 8, further comprising:
bringing the pharmaceutical composition into contact with a body,
wherein the bringing into contact is carried out at least one of orally, via an injection, topically, vaginally, rectally, and nasally.

22. A kit comprising:
at least one of at least one compound as recited in claim 1 and the pharmaceutical composition as recited in claim 8; and
information to bring at least one of the at least one compound and the pharmaceutical composition into contact with a body.

23. A method of using the kit as recited in claim 22 as a prophylaxis or to treat at least one of a neurodegenerative disease, a bacterial infectious disease, and a tumor, the method comprising:
providing the kit as recited in claim 22; and
using the kit as a prophylaxis or for a treatment of at least one of a neurodegenerative disease, a bacterial infectious disease, and a tumor.
